(19) United States
(12) Patent Application Publication
(10) Pub. No.: US 2018/0353461 A1
(43) Pub. Date: Dec. 13, 2018

(54) USE OF CANNABINOIDS AS CERAMIDE-GENERATING ANTICANCER AGENTS IN TUMORS OF THE HEMATOPOIETIC AND LYMPHOID TISSUES

(71) Applicant: Servicio Andaluz de Salud, Sevilla (ES)

(72) Inventors: José Antonio PÉREZ SIMÓN, Sevilla (ES); María Victoria BARBADO GONZÁLEZ, Sevilla (ES); María Teresa MEDRANO DOMÍNGUEZ, Sevilla (ES)

(21) Appl. No.: 15/533,337

(22) PCT Filed: Dec. 4, 2015
(86) PCT No.: PCT/EP2015/078669

§ 371 Date: Jun. 5, 2017

(30) Foreign Application Priority Data
Dec. 4, 2014 (EP) 14382495.1

(51) Int. Cl. A61K 31/352; A61K 31/5383; A61P 35/02; A61P 35/00

(52) U.S. Cl. CPC A61K 31/352; A61P 35/00; A61P 35/02; A61K 31/5383

(57) ABSTRACT

A ceramide-generating anticancer agent or treatment, and/or a ceramide degradation inhibitor, or a pharmaceutically acceptable salt or ester thereof, for use in the prevention, treatment, or amelioration of cancer or tumors of the hematopoietic and lymphoid tissues.

### FIELD OF THE INVENTION

The present invention relates to the field of Medicine, particularly to the medical treatments, and more specifically to the cannabinoids for use in the treatment of cancer and tumors of the hematopoietic and lymphoid tissues.

### BACKGROUND OF THE INVENTION

Multiple myeloma (MM) is the second most frequent hematologic malignancy. In the recent years, remarkable progresses have been achieved in the control of this disease, mostly due to the introduction of novel drugs, such as proteasome inhibitors or immunomodulatory drugs (IMIDs) in the therapeutic armamentarium. Nevertheless, it is still considered an incurable disease and, after a variable period of time, progression occurs and resistance finally emerges (1, 2). For this reason, great efforts are being devoted to discover novel drugs with increased efficacy and low toxicity profile which should display different mechanisms of action as compared to the currently available treatment options, so that they could be either combined or used as an alternative approach once resistance occurs.

Cannabinoids are the active components of Cannabis sativa linnaeus (marijuana) and their derivatives. Therapeutic interest on cannabinoids emerged after the discovery of an elaborate endocannabinoid physiological control system in humans (3, 4). The core of this endocannabinoid system consists of cannabinoid receptors (CBs). To date, two different cannabinoid receptors have been identified, name CB1 (5) and CB2 (6). The prior is extremely abundant in the peripheral and central nervous system, while CB2 is almost exclusively present in hematopoietic and immune cells (7) which also express CB1 albeit to a much lesser extent (8). However, it is not yet clear whether these receptors are the only ones capable of interacting with cannabinoids compounds (9).

In the last 20 years, different studies have reported that cannabinoids are relevant in many physiological functions, such as nociception, synaptic transmission or bone homeostasis among others (10). Moreover, there is increasing evidence supporting that they might be useful in the treatment of diseases such as glaucoma, multiple sclerosis, cardiovascular disorders, pain and neurodegenerative disorders (11-13). Moreover, cannabinoids are successful in mitigating nausea and vomiting associated with cancer treatment (14, 15). Nevertheless, the most increasing therapeutic interest in the cannabinoid research is currently their anti-tumor activity (16, 17). In this regard, some cannabinoids inhibit the proliferation of several tumour cells, such as glioma cell lines, both in vitro and in vivo (18-22). Different mechanisms of cannabinoid signal transduction have been suggested to justify this effect including pathways involved in proliferation, cell survival and apoptosis (23-26). In addition, cannabinoids might induce apoptosis by stimulating the synthesis of ceramides (18, 27-29).

As mentioned above, some hematopoietic cell subsets display high levels of CB2 particularly B-cells (7, 30). Despite this information, the effect of cannabinoids on the hematopoietic cells and lymphocytes has been investigated to a much lesser extent than in neuronal cells, so that most of their effects have been inferred from the studies on CB1 receptor. Therefore, many features of CB2 receptor function and regulation remain poorly characterized (31-34).

Considering previous evidences, the inventors propose that cannabinoids could be a good therapeutic candidate for use in the treatment of tumors of the hematopoietic and lymphoid tissues, and specifically for use in the treatment of multiple myeloma (MM) and acute myeloid leukemia (AML).

### DESCRIPTION OF THE INVENTION

The inventors observed a remarkable decrease in myelomatous cells viability, both cell lines and primary plasma cells from myeloma patients, upon exposure to cannabinoids. By contrast, cannabinoids had no effect on normal healthy cells, including hematopoietic progenitor stem cells.

Accordingly, cannabinoids exhibit a very selective anti-myeloma effect. The heterogeneity of the CBs pattern in hematopoietic cells is far too complex to conclude a clear relationship between receptor expression and sensitivity to the drug.

The analysis of PARP and Caspases reveals that cannabinoids trigger the two classical apoptotic signaling pathways, extrinsic and intrinsic, as well as the Casp-2-triggered pathway. The processing of Casp-2, -8 and -9 are observed as early as 2 h after exposure to the drug, but the activation over time of Casp-2 is notably stronger and, moreover, the up-regulation of cleaved forms of Casp-2 is parallel to the up-regulation of the PARP-fragment, which suggests a dominant role of Casp-2. In this regard, increasing evidence suggests that Casp-2 can function as initiator of apoptosis (37-38; Shi, 2005; Lava et al., 2012), It has been shown that involvement of Casp-2 occurs upstream of both mitochondrial damage and Casp-3 activation and, under stress conditions, a rapid up-regulation of Casp-2 occurs before induction of apoptosis (43). In multiple myeloma it has also been described that its activation occurs upstream of Casp-9 upon treatment with bortezomib (42). In accordance with these studies, our results suggest that Casp-2 plays a central role in the cannabinoid-induced apoptosis. Remarkably, Casp-2 is localized into ER membrane (43) and early studies have shown that it can be activated through ER-stress (42-43; 45-46). The ER responds to the burden of unfolded proteins (ER stress) by activating the unfolded protein response (UPR). UPR activation increases ER abundance to match needs by mediating expansion of the ER membrane. As such, the UPR establishes and maintains homeostasis (Peter Walter and David Ron. The Unfolded Protein Response: From Stress Pathway to Homeostatic Regulation Science 334, 1081 (2011). Due to the production of high levels of monoclonal immunoglobulins, myelomatous cells present a highly-developed ER and this makes MM cells particularly vulnerable to perturbations on protein metabolism (White-Gilbertson et al., 2013). In fact, the ER from myelomatous cells was referred as their “Achilles heel” (Pelletier et al., 2006), (Oslowski and Urano, 2011).

Therefore, to know the involvement of ER-stress in cannabinoid-induced apoptosis in myelomatous cells, we explored the expression profile of different proteins related to UPR before and after exposure to the drugs. Remarkably, myeloma cells lines display a high expression levels of UPR proteins, including CHOP, p-IRE1 and ATF-4, and this expression decreased upon exposure to cannabinoids. This result is in contrast to our own experience using leukemic cell cell lines (unpublished data) and also to the experience reported by other authors using glioma cell lines. It could be speculated that either cannabinoids attenuate ER stress or, on the contrary, impair the ability of MM cells to use the cytoprotective role of the UPR (Carrasco et al., Cancer Cell 11, 349 (2007; I. Papandreou et al., Blood 117, 1311 (2011). Finally, it is worth keeping in mind that UPR may provide opposing signals between cytoprotection and apoptosis.

Remarkably, RE is involved in the novo synthesis of ceramides (47, 48) which play a major regulatory role in apoptosis, enhancing the signaling events that drive apoptosis (49-52). In the current study the inventors found that cannabinoids upregulate SPT and increase the levels of ceramides, as confirmed by immunohistochemistry assays. In addition, the inhibition of SPT by myriocin abolished fragmentation of PARP, thus confirming the central role of ceramide synthesis in the pro-apoptotic effect of cannabinoids. Our results are consistent with previous studies conducted in glioma cells showing that cannabinoids may induce accumulation of ceramide mainly by de novo synthesis (18, 27-29, 53).

The present study represents the first evidence of a successful in vivo treatment with a ceramide-generating anticancer agent or treatment or a ceramide degradation inhibitor in a murine xenograft model of multiple myeloma. The inventors found that a ceramide-generating anticancer agent or treatment or a ceramide degradation inhibitor induce an impressive tumor growth inhibition, without mediating overt toxicity. The results collectively suggest that a a ceramide-generating anticancer agent or treatment or a ceramide degradation inhibitor are a promising treatment for MM.

In one aspect, the present invention relates to the use of:
a) a ceramide-generating anticancer agent or treatment; and/or
b) a ceramide degradation inhibitor;
   or a pharmaceutically acceptable salt or ester thereof, for use in the prevention, treatment, or amelioration of cancer or tumors of the hematopoietic and lymphoid tissues.

In a preferred embodiment of this aspect, the ceramide-generating anticancer agent or treatment or the ceramide degradation inhibitor is selected form the list consisting on anandamide, ceramidase inhibitors, chemotherapeutic agents, fas ligand, endotoxin, homocysteine, heat, gamma interferon, ionizing radiation, matrix metalloproteinases, reactive oxygen species, tetrahydrocannabinol and other cannabinoids, TNF-alpha, 1,25 dihydroxy vitamin D, or combinations thereof.

The antineoplasic properties of the cannabinoids were not addressed until late 90s in studies conducted in glioma (35) and breast cancer (36) cell lines. The inventors report for the first time on the anti-tumor potential of a ceramide-generating anticancer agent or treatment or a ceramide degradation inhibitor for the treatment of multiple myeloma.

In another preferred embodiment, the ceramide-generating anticancer agent or treatment, and/or the ceramide degradation inhibitor is a cannabinoid.

In a another preferred embodiment, the cannabinoid is selected from the group consisting of: HU-308; JWH-133; L-759, 633; PRS 211,375 (Cannabinor); AM-1241; JWH-015; L-759, 656; GW-842, 166X; GP-1 a; THC (Tetrahidrocannabinol); HU-210; L-759, 656; WIN 55,212-2; CP 55940; CRA-13; SAB-378; JWH-018 (or AM-678); CP 50,556-1 (levonantradol), or combinations thereof.

In another more preferred embodiment, the cannabinoid is WIN 55,212-2 or a pharmaceutically acceptable salt or ester thereof.

In another more preferred embodiment, the cannabinoid is JWH-133 or a pharmaceutically acceptable salt or ester thereof.

In another preferred embodiment, the cancer or tumors of the hematopoietic and lymphoid tissues is selected from the group consisting of: acute lymphoblastic leukemia (ALL), Acute myelogenous (or myeloid) leukemia (AML), Chronic lymphocytic leukemia (CLL), Acute monocytic leukemia (AMoL), Hodgkin's lymphomas (all four subtypes), Non-Hodgkin's lymphomas (all subtypes), myelomas (multiple myeloma), or combinations thereof.

In another more preferred embodiment, the cancer or tumor of the hematopoietic and lymphoid tissues is the acute myelogenous (or myeloid) leukemia (AML).

In another more preferred embodiment, the cancer or tumor of the hematopoietic and lymphoid tissues is the acute multiple myeloma (MM).

In another preferred embodiment, the cannabionid agent is natural and is selected from the group consisting of: cannabigerol-type agent (CBG), cannabichromene-type agent (CBC), cannabinodiol-type agents (CBND), tetrahydrocannabinol-type agents (THC), cannabinol-type agents (CBN), cannabitriol agents (CBT), cannabielsoin agent (CBE), isocannabinoids agents, cannabiciclol-type agents (CBL), cannabicitran-type agents (CBT), cannacichromanone-type agents (CBCN), or combinations thereof.

In another preferred embodiment, the natural cannabinoid agent is a cannabigerol-type agent (CBG), and is selected from the group consisting of: cannabigerol (E)-CBG-C5; cannabigerol monomethyl ether (E)-CBGM-C₅ A; cannabinerolic acid A (Z)-CBGA-C₅; cannabigerovarine (E)-CBGV-C₃; cannabigerolic acid A (E)-CBGA-C₅; cannabigerolic acid A monomethyl ether (E)-CBGAM-C₅; cannabigerovarinic acid A (E)-CBGVA-C₃, or combinations thereof. In another preferred embodiment, the natural cannabinoid agent is a cannabichromene-type agent (BCC), and is selected from the group consisting of: (±)-cannabichromene CBC-C₅; (±)-cannabichromene acid A CBC-C₅ A; (±)-cannabichromevarin CBCV-C₃; (±)-cannabichromevarinic acid A CBCV-C₃ A, or combinations thereof.

In another preferred embodiment, the natural cannabinoid agent is a cannabidiol-type agent (CBD), and is selected from the group consisting of: (-)-cannabidiol CBD-C₅; cannabidiol momometil ether CBDM-C₅; cannabidiol-C4 CBD-C₄; (-)-cannabidivarine CBDV-C₃; cannabidiorcol CBD-C₁; cannabidiolic acid CBDA-C₅; cannabidivarinic acid CBDVA-C₃, or combinations thereof.

In another preferred embodiment, the natural cannabinoid agent is a cannabinodiol-type agent (CBND), and is selected from the group consisting of: cannabinodiol CBND-C₅; cannabinodivarine CBND-C₃, or combinations thereof.

In another preferred embodiment, the natural cannabionoid agent is a tetrahydrocannabinol-type agent (THC), and is selected from the group consisting of: Δ⁹-Tetrahydrocannabinol Δ⁹-THC-C₅; Δ⁹-Tetrahydrocannabinol-C₄ Δ⁹-THC-C₄; Δ⁹-Tetrahydrocannabivarine Δ⁹-THC-C₃; Δ⁹-Tetrahydrocannabiorcol Δ⁹-THCO-C₁; Δ⁹-Tetrahydrocannabinolic acid A Δ⁹-THCA-C₅ A; Δ⁹-Tetrahydrocannabinolic acid B Δ⁹-THCA-C₅ B; Δ⁹-tetrahydrocannabinolic-C₄A and/or B Δ⁹-THCA-C₄ and/or B; Δ⁹-tetrahydrocannabivarinic acid A Δ⁹-THCA-C₃ A; Δ⁹-tetrahydrocannabiorcolic A and/or B Δ⁹-THCOA-C₁ A and/or B; (-)-Δ⁸-trans (6aR,10aR)-Δ⁸-tetrahydrocannabinol Δ⁸-THCA-C₅; (-)-Δ⁸-trans-(6aR,10aR)-tetrahydrocannabinolic acid Δ⁸-THCA-C₅ A; (-)-(6aS,10aR) Δ⁹-tetrahydrocannabinol (-)-cis-Δ⁹-THC-C₅, or combinations thereof.

In another preferred embodiment, the natural cannabionoid agent is a cannabinol-type agent (CBN), and is selected from the group consisting of: cannabinol CBN-C₅; cannabinol-C₄ CBN-C₄; cannabivarin CBN-C₃; cannabinol-C₂ CBN-C₂; cannabiorcol CBN-C₁; cannabinolic acid A CBNA-C₅, or combinations thereof.

In another preferred embodiment, the natural cannabionoid agent is a cannabitriol-type agent (CBT), and is selected from the group consisting of: (-)-(9R,10R)-trans-cannabitriol (-)-trans-CBT-C₅; (+) (9S,10S)-Cannabitriol (+)-trans-CBT-C₅; (±)-(9R,10S/9S,10R)-Cannabitriol (±)-cis-CBT-C₅; (-)-(9R,10R)-trans-10-O-Ethylcan nabitriol (-)-Trans-CBT-OEt-C₅; (±)-(9R,10R/9S,10S)-Cannabitriol-C₃ (±)-transCBT-C₃; 8,9-Dihydroxy-Δ^{6a(10a)}-tetrahydrocannabinol 8,9-Di-OH-CBT-C₅; cannabidiolic acid A cannabitriol ester CBDA-C₅ 9-OH-CBT-C₅ ester; (-)-(6aR,9S,10S,10aR)-9,10-Dihydroxy-hexahidrocannabinol, Cannabiripsol Cannabiripsol-C₅; (-)-7a,10^{a} trihydroxy Δ⁹-tetrahydrocannabinol (-)-Cannabitetrol; 10-Oxo-Δ^{6a(10a)} tetrahydrocannabinol OTHC, or combinations thereof.

In another preferred embodiment, the natural cannabinoid agent is a cannabinol-type agent cannabielsoin (CBE), and is selected from the group consisting of: (5AS,6S,9R,9aR)-Cannabielsoin CBE-C₅; (5AS,6S,9R,9aR)-C3-Cannabielsoin CBE-C₃; (5AS,6S,9R,9aR) Cannabielsoic acid A CBEA-C₅ A; (5AS,6S,9R,9aR)-Cannabielsoic acid CBEA-C5 B CBEA-C₅ B; Cannabiglendol-C₃ OH-iso-HHCV-C₃; Dehidrocannabifuran DCBF-C₅; Cannabifuran CBF-C₅, or combinations thereof.

In another preferred embodiment, the natural cannabinoid agent is an isocannabinoid-type agent, and is selected from the group consisting of: (-)-Δ⁷-trans-(1R,3R,6R)-Isotetrahydrocannabinol; (±) cis-Δ⁷-1,2 (1R,3R,6S/1S,3S,6R)-Isotetrahydrocannabivarin, (±) cis-Δ7-1,2 (1R,3R,6S/1S,3S,6R)-Isotetrahydrocannabivarin, or combinations thereof.

In another preferred embodiment, the natural cannabinoid agent is a cannabiciclol-type agent (CBL), and is selected from the group consisting of: (±)-(1aS,3aR,8bR,8CR) -Cannabiciclol CBL-C₅; (±)-(1aS,3aR,8bR,8CR)- Cannabiciclolic acid CBLA-C₅ A; (±)-(1aS,3aR,8bR,8CR)-Cannabiciclovarin CBLV-C₃, or combinations thereof.

In another preferred embodiment, the natural cannabinoid agent is a cannabicitran-type agent (CBL), and is selected from the group consisting of: Cannabicitran CBT-C₅, or combination thereof.

In another preferred embodiment, the natural cannabinoid is a natural cannabichromaneme-type agent, and is selected from the group consisting of: cannabichromaneme CBCN-C₅; cannabichromaneme -C₃ CBCN-C₃; cannabicoumaronone CBCON-C₅, or combination thereof.

In a second aspect, the invention relates to a composition, hereinafter composition of the invention, comprising a ceramide-generating anticancer agent or treatment, and/or the ceramide degradation inhibitor as described in the first aspect of the invention, for use in the prevention, treatment, or amelioration of cancer or tumors of the hematopoietic and lymphoid tissues.

In a preferred embodiment, the composition of the invention further comprises another active ingredient. In another preferred embodiment, the composition of the invention further comprises a pharmaceutically acceptable carrier. In a preferred embodiment, the composition of the invention is a pharmaceutical composition

In a third aspect, the invention relates to a pharmaceutical form, in the following the pharmaceutical form of the invention, comprising the composition of the invention, for use in the prevention, treatment, or amelioration of cancer or tumors of the hematopoietic and lymphoid tissues.

In a fourth aspect, the invention relates to a method of selecting a ceramide-generating anticancer agent or treatment and/or a ceramide degradation inhibitor useful in the prevention, treatment, or amelioration of cancer or tumors of the hematopoietic and lymphoid tissues as defined in the first, second and/or third aspect of the invention, comprising:
a) contacting the test compound with a hematopoietic and/or lymphoid cell or cell line, and
b) detecting the expression of serine palmitoyltransferase (SPT).

In a preferred embodiment, the cell or cell line is a hematopoietic or lymphoid cell or cell line. Wherein the SPT expression level in the cell or cell line is enhanced upon incubation with the test compound in comparison to a control cell or cell line that is not in contact with the test compound, is considered that this test compound is a ceramide-generating anticancer agent or treatment and/or a ceramide degradation inhibitor useful in the prevention, treatment, or amelioration of cancer or tumors of the hematopoietic and lymphoid tissues. The cell or cell line can be a normal cell or cell line, preferably a hematopoietic or lymphoid cell or cell line. Also, in another preferred embodiment the cell or cell line is obtained from patients with an hematopoietic and/or lymphoid cancer, for example but not limited to LMA patients, The control cell or cell line can be, for example but without limitation, the same cell or cell line of the step a) that is not in contact with the test compound.

In a fifth aspect, the invention relates to a method of selecting a ceramide-generating anticancer agent or treatment and/or a ceramide degradation inhibitor useful in the prevention, treatment, or amelioration of cancer or tumors of the hematopoietic and lymphoid tissues as defined tissues in the first, second and/or third aspect of the invention comprising:
a) contacting the test compound with a hematopoietic and/or lymphoid cell or cell line, and
b) detecting the expression of ceramide by immunohistochemistry.

In a preferred embodiment, the cell or cell line is a hematopoietic or lymphoid cell or cell line. Wherein the SPT expression level in the cell or cell line is enhanced upon incubation with the test compound in comparison to a control cell or cell line that is not in contact with the test compound, is considered that this test compound is a ceramide-generating anticancer agent or treatment and/or a ceramide degradation inhibitor useful in the prevention, treatment, or amelioration of cancer or tumors of the hematopoietic and lymphoid tissues. The cell or cell line could be a normal cell or cell line, preferably a hematopoietic or lymphoid cell or cell line. Also, in another preferred embodiment the cell or cell line is obtained from patients with an hematopoietic and/or lymphoid cancer, for example but not limited to LMA patients, The control cell or cell line can be, for example but without limitation, the same cell or cell line of the step a) that is not in contact with the test compound.

Along the description and claims, the word “comprises” and variants thereof do not intend to exclude other technical features, supplements, components or steps. For persons skilled in the art, other objects, advantages and features of the invention will be understood in part from the description and in part from the practice of the invention. The following examples and drawings are provided by way of illustration and they are not meant to limit the present invention.

### DESCRIPTION OF THE FIGURES

FIG. 1. Win-55 and JWH-133 have antiproliferative effects on MM cell lines. Analysis of cell viability in myeloma cell lines after incubation with cannabinoids as assessed by MTT. (A) Cells were treated with increasing concentrations of the cannabinoid WIN-55. (B) Cells were incubated at increasing doses of the cannabinoid JWH-133. The average proliferation values of control untreated samples were taken as 100%. Data are mean plus or minus SD of quadruplicates of an experiment that was repeated at least three times. Asterick indicates significant differences at p value ≤0.05.

FIG. 2. Cannabinoids reduce the cell viability of primary plasma cells from MM patients, but do not affect normal residual cell populations. The analysis were performed by flow cytometry. (A) Cells were treated with increasing concentrations of the cannabinoid WIN-55. (B) Cells were incubated at increasing doses of the cannabinoid JWH-133. The dot plots correspond to highest dose tested of WIN-55 (in A) and JWH-133 (in B). Plasma cells are identified as CD38+, lymphocytic cells as CD45+ and granulomonocytic cells as CD64+. The average proliferation values of control untreated samples were taken as 100%. Data are mean plus or minus SD of quadruplicates of an experiment that was repeated at least three times.

FIG. 3. Cell viability of CD34+ hematopoietic stem cells remains unaffected after cannabinoid treatment, Stem cells were treated with WIN-55 (left panel) or JWH-133 (right panel) and cell viability was analyzed using MTT assay. The average proliferation values of control untreated samples were taken as 100%. Data are mean plus or minus SD of quadruplicates of an experiment that was repeated at least three times. Asterick indicates significant differences at p value ≤0.05.

FIG. 4. Cannabinoids affect B-cells while T-cells viability is maintained. (A) T-, in left panel, and B-cells, in right panel, were treated with WIN-55. (B) T-, in left panel, and B-cells, in right panel, were treated with JWH-133, and subsequently analyzed by MTT assays. The average proliferation values of control untreated samples were taken as 100%. Data are mean plus or minus SD of quadruplicates of an experiment that was repeated at least three times. Asterick indicates significant differences at p value ≤0.05.

FIG. 5. Profile of CBs expression in MM cell lines and primary cells is very complex and heterogeneous. We analyzed by Western blot the expression pattern of cannabinoid receptors in primary cells from healthy donors and multiple myeloma cell lines. (A) Expression profile of CB1 in hematopoietic stem cells, B- and T-cells, in left panel, and MM cell lines in right panel. (B) Expression profile for CB2.

FIG. 6. The effect of cannabinoids in MM cells is mediated by apoptotic processes. Western blot analysis of the protein expression in U266 cells treated with WIN-55 during 0, 2, 6, 18 and 24 hours. The expression of tubulin was used as loading control. (A) Profile time-course of the expression of the executioner caspase, Casp-3 and its substrate, PARP. (B) Expression of the main initiator caspases, Casp-9, Casp-2 and Casp-8. (C) Expression of pro-apoptotic Bak and Bax, and anti-apoptotic regulator proteins Mcl-1 and Bcl-xL.

FIG. 7. The main transduction signal triggered by WIN-55 is the Akt/PKB pathway. U266 cells were treated at the indicated times on the top of panel with WIN-55 50 uM, and the signaling pathways, MAPKs and Akt were analyzed.

FIG. 8. Ceramides play a crucial role in the cannabinoid-induced apoptosis. (A) Western blot analysis of the expression of serine-palmitoyl-transferase, limiting enzyme of the de novo ceramide synthesis, upon exposure to cannabinoid WIN-55 50 uM. (B) Photomicrograph of staining of ceramides in U266 cells untreated (left) and treated with WIN-55 50 um for 18 hours.(C) Expression of PARP, full-length and fragmented, in untreated cells (left lane), cannabinoid-treated cells (central lane) and cells co-incubated with cannabinoid and SPT-inhibitor, myriocin, at 6 hours.

FIG. 9. Win-55 attenuates the response to ER-stress in myelomatous cells. Expression analysis by Western blot of regulator proteins of ER-stress in U266 cells treated with WIN-55 50 uM.

FIG. 10. Cannabinoid treatment promotes an early loss of potential of mitochondrial membrane. We evaluated the potential of mitochondrial membrane potential in U266 cells after treatment with WIN, at indicated times, using TMRE assay.

FIG. 11. Cannabinoid inhibit tumor growth in vivo. To check the antimyeloma effect of the cannabinoid WIN-55 on MM cells in vivo we inoculated NSG mice with U266 cells. Tumor diameters were measured and the tumor volume was estimated as volume of an ellipse.

FIG. 12. Selective antiproliferative effect of cannabinoid WIN-55 on cell viability. Incubation with WIN-55 for 18 h significantly reduced the cell viability of KG1 (acute myelogenous leukemia) and HL60 (Human promyelocytic leukemia cells) cell lines tested as compared to untreated control cells. Although both cell lines were sensitive to treatment with WIN-55, HL60 was the most sensitive. The cell viability decrease occurs from 500 uM with statistically significant data. The average proliferation values of control untreated samples were taken as 100%. Data are mean plus or minus SD of quadruplicates of an experiment that was repeated at least three times. Asterick indicates significant differences at p value ≤0.05.

FIG. 13. Sensitivity pattern to cannabinoid JWH-133. The cell viability decrease occurs from 500 uM with statistically significant data. The average proliferation values of control untreated samples were taken as 100%. Data are mean plus or minus SD of quadruplicates of an experiment that was repeated at least three times. Asterick indicates significant differences at p value ≤0.05.

FIG. 14. Cannabinoids reduce the cell viability of blasts from AML patients, but do not affect normal cell populations Cell viability of CD34+ hematopoietic stem cells remains unaffected after cannabinoid treatment. Stem cells were treated with WIN-55 or JWH-133 and cell viability was analyzed using MTT assay. The average proliferation values of control untreated samples were taken as 100%. Data are mean plus or minus SD of quadruplicates of an experiment that was repeated at least three times. Asterick indicates significant differences at p value ≤0.05.

FIG. 15. Profile of CBs expression in AML cell lines and primary cells is very complex and heterogeneous. We analyzed by Western blot the expression pattern of cannabinoid receptors in primary cells from healthy donors and AML cell lines.

The effect of cannabinoids in AML cells is mediated by apoptotic processes. Western blot analysis of the protein expression in HL60 cells treated with WIN-55 50 uM during 0, 2, 6, 18 and 24 hours. The expression of tubulin was used as loading control.

FIG. 16. Profile time-course of the expression of the executioner caspase, Casp-3 and its substrate, PARP. Expression of the main initiator caspases, Casp-9, Casp-2 and Casp-8.

FIG. 17. The main transduction signal triggered by WIN-55 is the Akt/PKB pathway. HL60 cells were treated at the indicated times on the top of panel with WIN-55 50 uM, and the signaling pathways, MAPKs (p-JNK, p-Erk1/2 and p-p38) and Akt were analyzed.

FIG. 18. Ceramides play a crucial role in the cannabinoid-induced apoptosis. (A) Photomicrograph of staining of ceramides in HL60 cells untreated (left) and treated with WIN-55 50 um for 18 hours (B) Expression of active form of PARP, in untreated cells (left lane), cannabinoid-treated cells (central lane) and cells co-incubated with cannabinoid and SPT-inhibitor, myriocin, at 2 hours.

FIG. 19. Win-55 increase the ER-stress in AML cells. Expression analysis by Western blot of regulator proteins of ER-stress in HL60 cells treated with WIN-55 50 uM.

FIG. 20. Cannabinoid treatment promotes an early loss of potential of mitochondrial membrane. We evaluated the potential of mitochondrial membrane potential in HL60 cells after treatment with WIN, at indicated times, using TMRE assay by floy citometry. Also it was evaluated the ROS increase by MitoSOX assay.

FIG. 21. Cannabinoid inhibit tumor growth in vivo. Anti-tumor effect of the cannabinoids in vivo, using a model of human AML (HL60) xenografted in immunodeficient mice NOD/SCID.

### EXAMPLES

The following specific examples provided in this patent document serve to illustrate the nature of the present invention. These examples are included only for illustrative purposes and must not be interpreted as limiting to the invention claimed herein. Therefore, the examples described below illustrate the invention without limiting the field of application thereof.

### Example 1

### Materials and Methods

### Ethics Statement

All research involving human materials was approved by the Ethical Committee for Clinical Research (CEIC) of the University Hospital Virgen del Rocío, and informed consents were obtained from all donors in accordance with the Declaration of Helsinki. Animal experimentations described in the present paper have been conducted in accordance with accepted standards of animal care and in accordance with the Spanish regulations for the welfare of animals used in studies of experimental neoplasia, and the study was approved by our institutional committee on animal care.

### Cell Cultures and Primary Cells

Human multiple myeloma cell lines MM1.S, MM1.R, RPMI-8226 (RPMI) and U266 were purchased from American Type Culture Collection [(ATCC), LGC Standards, Manassas, USA]. Human primary cells were obtained from MM patients' bone marrow (BM) and healthy donors' peripheral blood (PB). PB samples were collected from buffy coats and leukapheresis products, previously mobilized with granulocyte colony stimulating factor (G-CSF). Hematopoietic progenitor cells (CD34+) were isolated from leukaphesesis samples, and B (CD19+) and T (CD3+) lymphocytes from buffy coats by positive immunomagnetic separation in the AutoMACS pro separator (Miltenyi Biotec, Bergisch Gladbach, Germany). For this purpose CD34-, CD19- and CD3-MACS microbead Human Kit (Miltenyi Biotec, Bergisch Gladbach, Germany). The purity of all isolated cells was higher than 95% in all cases. BM samples were obtained from five MM patients. In all cases, BM plasma cell infiltration was greater than 30%. The different cell subpopuplations from MM patients BM were not separated, but they were identified by flow cytometry using a suitable combination of antibodies: anti-CD64-FITC, anti-CD34-PE, anti-CD56-APC, anti-CD38-APC-H7, and anti-CD45-Pacific Blue (BD Biosciences, San Jose, Calif.).

All cell lines and primary cells were maintained in RPMI 1640 medium (Gibco™, Invitrogen, Barcelona, Spain), with 2 mM L-glutamine, 100 IU/ml penicillin and 100 ug/ml streptomycin (all from Sigma-Aldrich, St. Louis, Mo., USA), and supplemented with 10% (multiple myeloma cell lines) or 20% (human primary cells) fetal bovine serum [(FBS), Thermo Fisher Scientific (Waltham, Mass., USA)]. Cells were maintained at 37° C. in a humidified atmosphere in the presence of 5% CO2/95% air. Cells were transferred to a serum-free medium 30 min before performing the different treatments and were maintained in this medium for the rest of the experiment.

### Drugs and Treatments

The cannabinoid agonists WIN-55,212-2 mesylate ((R)-(+)-[2,3-dihydro-5-methyl-3-(4-morpholinylmethyl)-pyrrolo-[1,2,3]-1,4-benzoxazin-6-yl]-1-naphthalenylmethanone mesylate) and JWH-133 ((6aR,10aR)-3-(1,1-Dimethylbutyl)-6a,7,10,10a-tetrahydro-6,6,9-trimethyl-6H-dibenzo [b,d] pyran) were purchased from Tocris Bioscience (Bristol, UK). Win-55, 212-2 and JWH-133 were reconstituted with 100 mM and 50 mM DMSO (Sigma-Aldrich) in distilled water, respectively.

Myriocin (ISP-1), the inhibitor of the serin-palmitoyl-transferase (SPT) enzyme, was obtained from Enzo Life Sciences (Lausen, Switzerland). Both antagonists and inhibitor were reconstituted in 100 mM DMSO.

Except if otherwise indicated, for the viability assays primary cells were incubated 18 hours, with vehicle (with <0.15% DMSO) or cannabinoid. Meanwhile, the time course assays cells were performed at 0, 2, 6, 18 and 24 h. For the analysis of SPT activity, cells were co-incubated for the first 6 hours with the SPT inhibitor, myriocin, and the cannabinoid, WIN-55 or JWH-133.

The working concentrations of cannabinoids were determined by dose-response curves (data not shown) and selected within the linear range between observed safe and hazardous levels. These doses were used as follow: 0.5, 1, 10, 20 and 50 uM for WIN-55, and 0.05, 0.1, 0.2, 0.5 and 1 mM for JWH-133. In the same way, optimal doses of SPT inhibitor were defined. Control condition was treated with PRMI medium with the highest percentage of DMSO used in each trial. In all experiments the final concentration of DMSO contained in the vehicle or drug never exceeded 0.15% (v/v), a non-cytotoxic concentration.

### Reagents and Antibodies

Antibodies for detection CB1- and CB2-receptor, caspases-2, -3, -8, -9, and phosphorylated forms of signaling pathways molecules Akt, Erk 1/2, p38MAPK, JNK and SPT were obtained from Abcam Company (Cambridge, UK). For Western Blot analyses, as a loading control were used anti-beta-actin and anti-beta-tubulin (Sigma-Aldrich). Antibodies against to key apoptotic signaling proteins PARP, Mcl-1, Bcl-xL, Bax and Bak were from BD Biosciences. All used secondary antibodies were Horseradish Peroxidase (HRP)-conjugated (Jackson ImmunoResearch Labs., Pa., USA), and produced in donkey to avoid potential cross-reactivity when multiple-probings were performed.

### Cell Viability Analysis

Cell viability of cell lines and CD34+, CD19+ and CD3+ cells was measured using the Cell Counting Kit-8 (Dojindo Molecular Technologies, Gaithersburg, Md.). This assay is based on the reduction of salt WST-8 by a mitochondrial dehydrogenase in viable cells, resulting in a colored formazan product that can be measured spectrometrically at 450 nm. Briefly, 5×10e5 cells per well were seeded onto a 96-well plate and cultured in triplicate in presence or absence of the different selected concentrations of WIN-55 or JWH-133. Primary cells were treated for 12 and 18 hours, and cell lines for 12, 18 and 48 h. Moreover, cells treated with DMSO (<0.15%) in RPMI medium were used as untreated controls. After treatment, 10 ul salt WST-8 [2-(4-lodophenyl)-3-(4-nitrophenyl)-5-(2,4-disulfophenyl)-2H-tetrazolium] was added to each well and incubated for an additional 2 h at 37° C., and then the absorbance of generated formazan was measured in a plate reader Multiskan™ Go Microplate (Thermo Fisher Scientific, Waltham, Mass., USA).

Further, cell viability of bone marrow cell populations from patients was assessed by flow cytometry using 7-amino-actinomycin D [(7-AAD), BD Biosciences] together with a combination of monoclonal antibodies against myeloma-associated antigens (anti-CD56-APC, anti-CD45-Pacific Blue, and anti-CD38-APC-H7 [BD Biosciences]) and antibodies to discriminate the granulomonocytic (anti-CD64-FITC) and lymphocytic (anti-CD45-Pacific Blue) populations. Cells were incubated for 15 minutes at room temperature in the dark with suitable antibodies and 7-AAD, and after washing a total of 100 000 cells were acquired on FACSCanto II flow cytometer (BD Biosciences). Fluorescent intensity was quantified and analyzed using Infinicyt™ Software (Cytognos, Salamanca, Spain). Additionally, cell viability of MM cell lines and the peripheral blood CD34+, CD3+ and CD19+ cell populations from healthy donors was also analyzed by flow cytometry using 7-AAD, with the same protocol above described.

### Analysis of Mitochondrial Transmembrane Potential

The loss of mitochondrial transmembrane potential (Δψm), a marker of early apoptotic events, was determined by staining with TRME [(tetramethylrhodamine-ethyl-ester-perchlorate), Santa Cruz Biotechnology Inc.]. TMRE is a cell-permeable dye that becomes fluorescent once it is inside the mitochondria and, it can be detected using a fluorescent plate reader (Tecan). Briefly, cells (10e6 cells per assay) were treated with different concentrations of WIN-55 in presence or absence of SPT-inhibitor for 15, 30, 45 and 60 min at 37° C. After washing twice, cells were incubated for 20 min at 37° C. in the dark with 1 uM TMRE. The fluorescent intensity was read in the plate reader with excitation and emission wavelengths set at 485 and 538 nm respectively for red fluorescence. For each condition, triplicate samples (at least five times) were run, fluorescent readings were corrected for background. In all assays, CCCP (2-[2-(3-Chlorophenyl) hydrazinylyidene] propanedinitrile, a potent mitochondrial uncoupler, was used as a positive control of mitochondrial potential status.

### Protein Sample Preparation

Protein analyses were performed by Western blot in several sets of assays: expression pattern of CBs-receptors in MM cell lines and primary cells, and time course after treatment with cannabinoids in MM cell lines. Time-course assays were performed at 0, 2, 6, 18 and 24 hours. Anyway, obtaining cell lysates was conducted according to a protocol adapted to phosphorylation-dependent signaling, described by Gilbert et al. (2002). Briefly, 10e7 cells per condition were harvested at indicated times, washed with iced-cold PBS and lysate in isotonic lysis buffer 30 min keeping on ice. Isotonic lysis buffer contained 2% ASB-14 (CalbioChem, San Diego, Calif., USA),1% Nonidet P-40 (Ipegal®), 137 mM NaCl, 20mM Tris-HCl pH 7.5 at 4° C. (all from Sigma-Aldrich), 2 mM Na3VO4, 20 mM NaF (New England BioLabs Inc., Ipswich, Mass., USA), 2 mM DTT (AppliChem, Darmstadt, Germany), protease inhibitor cocktail (Roche Diagnostics GmbH, Mannheim, Germany), and 10 ul Nuclease Mix (Amersham GE Healthcare, Uppsala, Sweden). After lysis, samples were centrifuged at 13,000×g for 5 min at 4° C., and supernatants collected. Protein concentration in samples was determined by Pierce® Microplate BCA Protein Assay kit-Reducing Agent Compatible (Thermo Fisher Scientific). Before loading the samples in the gel, they were diluted in loading sample buffer (0.125 M Tris-HCl pH 6.8, 4% SDS, 20% glycerol, 0.02% Bromophenol Blue, 200 mM DTT) and the mixture was heated at 70° C. for 5 min in a thermoblock (LabNet International, N.J., USA).

### Western Blot Analysis

Samples (15-25 ug/lane) were subjected to SDS/PAGE in pre-cast polyacrylamide gels AnykD (Bio-Rad Laboratories, Hercules, CA, USA) under reducing and denaturing conditions. Electrophoretic run was performed at a constant current of 15 mA per gel in a Mini-Protean® Tetra Cell (Bio-Rad Laboratories). Once proteins were separated by electrophoresis, were transferred onto PVDF membranes using Trans-Blot® Turbo™ System (Bio-Rad Laboratories). Electrotransference was conducted at constant amperage of 1.2 mA and at up to 25 V of voltage. The blotted membranes were then blocked with 2% bovine serum albumin [(BSA), Santa Cruz Biotechnology] in Tween-Tris buffer saline (TTBS) for 30 min with gentle agitation, and incubated overnight at 4° C. with corresponding primary antibody in TTBS. After twice washing with TTBS, membranes were incubated for 1-2 h at room temperature with suitable conjugated-HRP secondary antibody, and subjected to chemiluminescence detection using Western Blotting Luminol Reagent (Santa Cruz Biotechnology). Loading controls were carried out with an anti-beta-actin and anti-beta-tubulin antibodies (Sigma-Aldrich). Blots were captured using a digital imaging system ImageQuant LAS 4000 (Amersham GE Healthcare) and transferred to a digital image processing software to analyze (Adobe Photoshop CS2, version 9.0., Adobe Systems Inc., San Jose, Calif.).

### Immunocytofluorescence Analysis of Culture Cells

After cannabinoids and/or antagonists or SPT inhibitor treatment, primary cells and cell lines were washed with PBS, collected and placed on slides (Menzel-Glaser, Thermo Fisher Scientific). Cells were fixed in cold 4% paraformaldehyde for 10 min, treated with sodium borohydride and, permeabilized in 0.02% Triton X-100 in PBS with 10% normal donkey serum for blocking. Then, cells were incubated with the anti-CB1 or anti-CB2 receptor antibodies, anti-active-caspase3, anti-ceramide in blocking buffer overnight at 4° C. in a humid chamber (Simport, Beloeil, QC, Canada). After washing with PBS, samples were further incubated for 90 min with Alexa 647- or Alexa 488—conjugated donkey secondary antibody. After twice washing, first with PBS and then with 50 mM Tris-HCl (pH 7.4), and were dipped in a solution of Sudan Black B (Sigma-Aldrich) in 70% methanol for 5 min. After immunocytochemistry, slides were mounted using Fluoromount Aqueous Mounting Medium (Sigma-Aldrich). Preparations were examined with an Olympus BX-61 microscope (Olympus, Hamburg, Germany) equipped with appropriate filters: FITC/Cy2 (green fluorophores) 460-490 nm bandpass excitation filter and 510-550 nm bandpass emission filter; rhodamine/Cy3 (red fluorophores) 541-551 nm excitation and 572-607 nm emission; and Cy5 (deep red fluorophore) 615-635 nm excitation and 655 nm longpass emission. Digital images were acquired with CellSens Dimension Software (Olympus) and contrasted with Photoshop CS2 Software (Adobe Systems Inc). There was no labeling when the primary antibody was omitted (data not shown).

### MM Xenografts

NOD/scid/IL-2R gammae null (NSG) mice were purchased from Charles River Laboratories International (L'Arbresle, France) and maintained with food and water ad libitum, under specific pathogen-free conditions. When mice were 8-9 weeks old, human tumor xenografts were induced by subcutaneous inoculation in suprascapular area of MM cell line U266 cells. To establish tumor, 5×10e6 cells were resuspended in 100 ul RPMI medium without serum and 100 ul of Matrigel (BD Biosciences). To easy visualization the hair was trimmed from the site of inoculation. Closely second week following implantation, when tumors became palpable (>0.5 cm), mice were randomly assigned to the following groups (10 mice per group): group 1 received intraperitoneally 5 mg/kg WIN-55,212-2 every 24 h, group 2 received the treatment every 48 hl and group 3 received vehicle. Moreover, other two groups left tumor-free served as a negative control, received the drug every 24 or 48 h each one. Tumor growth was assessed three times each week following tumor implantation. Two bisecting diameters of each tumor were measured with calipers, and the volume was calculated using the formula length x (width) e2×0.4 mm3. Animals were sacrificed when the tumor length or width reached 2 cm. Time to endpoint was defined as the time from the day of initiation of treatment (when one diameter reaches 5 mm) to death as a result of toxicity or tumor growth (when length or width reached 2 cm mice were sacrificed), or any other cause.

### Statistical Analyses

For all statistical analysis, SPSS software version 15.0 (Statistical Package for the Social Sciences, SPSS, Chicago, Ill., USA) was used and statistical significance was defined as P≤0.05. Error bars represent standard error of the mean (SEM). Data were analyzed using t-Student test.

### Results

### Antiproliferative Effect in MM (In Vitro Assays)

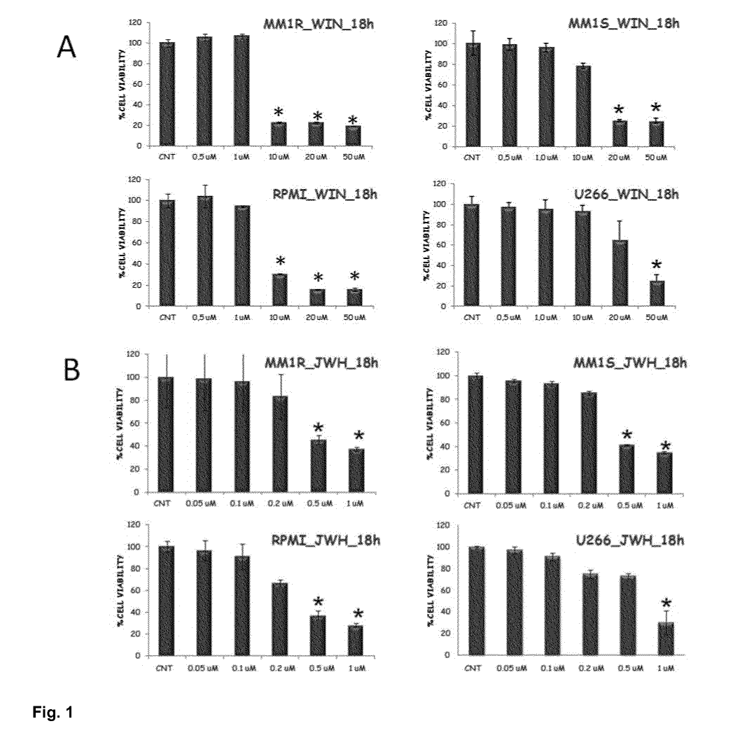

Cannabinoids have a highly selective antiproliferative effect. Firstly, we assessed the effect of cannabinoids WIN-55 and JWH-133 on cell viability. To this end, the cells were treated with increasing concentrations of cannabinoids, WIN-55 (0.5-50 uM) and JWH-133 (0.05-1 mM), for 18, 48 and 96 hours, and viability was analyzed by MTT assays and flow cytometry. We found that incubation with WIN-55 and JWH-133 for 18 h significantly reduced the cell viability of all MM cell lines tested as compared to untreated control cells (FIG. 1a, b). Although all cell lines were sensitive to treatment with WIN-55, MM1R and RPMI were the most sensitive (FIG. 1a). The sensitivity pattern to cannabinoid JWH-133 was similar, decreasing from MM1.R, RPMI, MM1.S to U266, (FIG. 1b). Similar results were obtained when the different cell lines were treated for 48 and 72 h (data not shown).

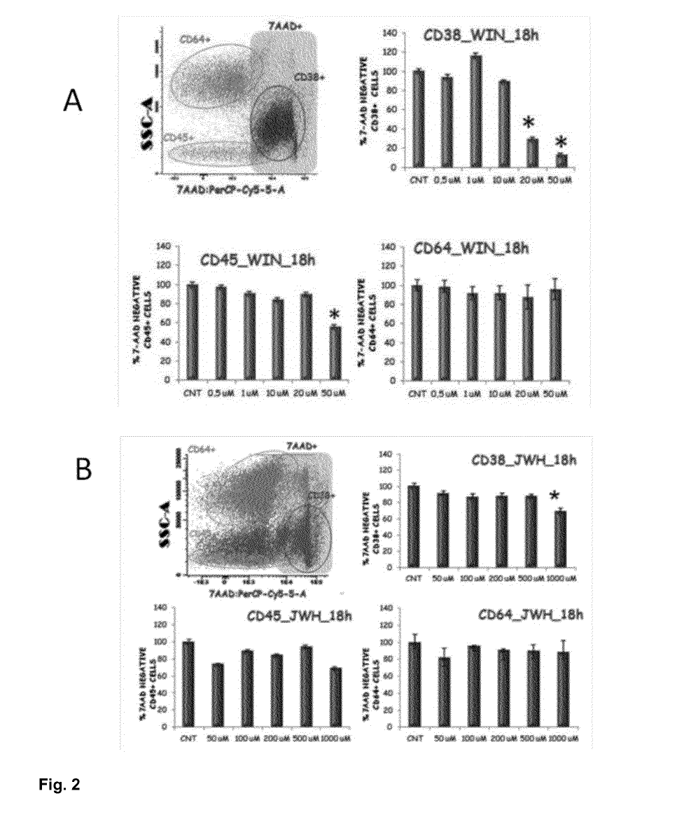

The effect of cannabinoids was further examined ex vivo in myeloma plasma cells (MPCs) isolated from bone marrow samples obtained from MM patients. As shown in FIG. 2a, upon treatment with WIN-55, MPCs showed a significant decrease in cell viability up to 85% at 50 uM as compared to control (vehicle-treated) cells. Similarly, exposure of MPCs to JHW-133 also led to a decreased viability, although less pronounced as compared to WIN-55 (FIG. 2b).

### Cell Viability

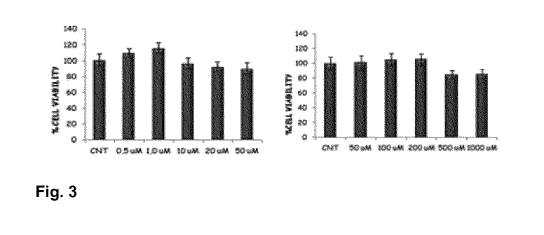

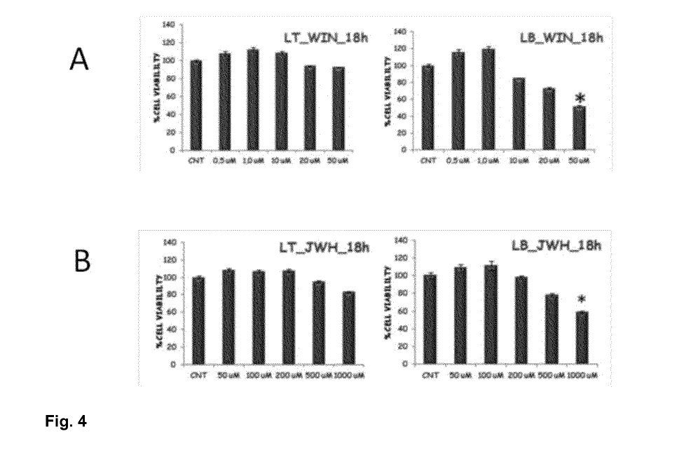

Once determined the antiproliferative effect of cannabinoids on tumor cells, both primary and cell lines, our aim was to test whether or not these drugs could also affect the viability of the counterpart normal cells, both normal residual populations from bone marrow samples obtained from MM patients as well as hematopoietic stem cells (CD34+) from healthy donors. Strikingly, we observed that the cell viability of hematopoietic stem cells remained unaffected. That is, neither WIN-55 nor JWH-133 exhibited antiproliferative effect at any tested dose on the CD34+ cells (FIG. 3). Granulocytes and lymphocytes, were also unaffected by both cannabinoids, except for the lymphocytes at the highest doses tested, 50 uM for WIN-55 and 1 mM for JWH-133 (FIG. 2a,b). As shown in FIG. 4, the cytotoxic effect of WIN-55 and JWH-133 in the lymphoid population was due to a decreased viability of B-cells, whereas viability of T-cells remained unaffected

Taken together, our results indicate that cannabinoids, WIN-55 and JWH-33, have a very selective effect as they induce apoptosis on myelomatous cells whereas viability of healthy cells, including hematopoietic precursor cells remained unaffected.

### Mechanism of Action

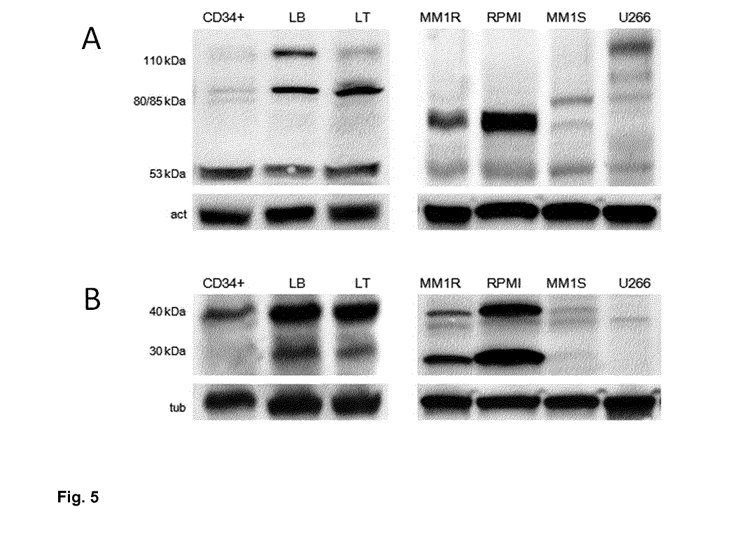

The expression pattern of cannabinoid receptors is very heterogeneous and does not correlate with the susceptibility to cannabinoids. To know whether this antiproliferative effect of cannabinoids is correlated with the content of the cannabinoid receptors, we evaluated the expression of both receptors in MM cell lines as well as in CD34+ cells and lymphocytes from healthy donors. We detected CB1 and CB2 expression in all cell subsets analyzed. Of note, both receptors showed multiple bands and a very heterogeneous pattern between the different cell subtypes analyzed (FIG. 5a, b). Regarding CB1 (FIG. 5a), the major band migrated at 53 kDa in accordance to the amino acid sequence corresponding to its monomeric form. Healthy cells expressed a higher level of the monomeric form of CB1 as compared to the myelomatous cells. The pattern of CB1 also exhibited the presence of others immunorreactive bands with high molecular weight, being particularly striking the bands migrating approximately at 80/85 and 100 kDa in the primary cells, and the band at ˜70 kDa in the MM cell lines. As shown in FIG. 5b, the expression profile of CB2 mainly exhibited two bands that were detected at 40 kDa, consistent with the predicted size of the CB2 protein, and other band at 30 kDa which was strongly immunorreactive in the MM1.R and RPMI cell lines. In addition, the lowest expression level of the CB2, both 30 and 40 kDa forms, was observed in hematopoietic stem cells and cell lines MM1S and U266.

In summary, our results show that the expression of CBs, both CB1 and CB2, is more heterogeneous than previously suspected, making difficult to conclude a clear association between receptor expression and sensitivity to cannabinoids. In spite of this lack of uniformity in the protein profiles of CBs, we found that the primary cells and cell lines more sensitive to the anti-proliferative effect of cannabinoids displayed higher levels of both receptors.

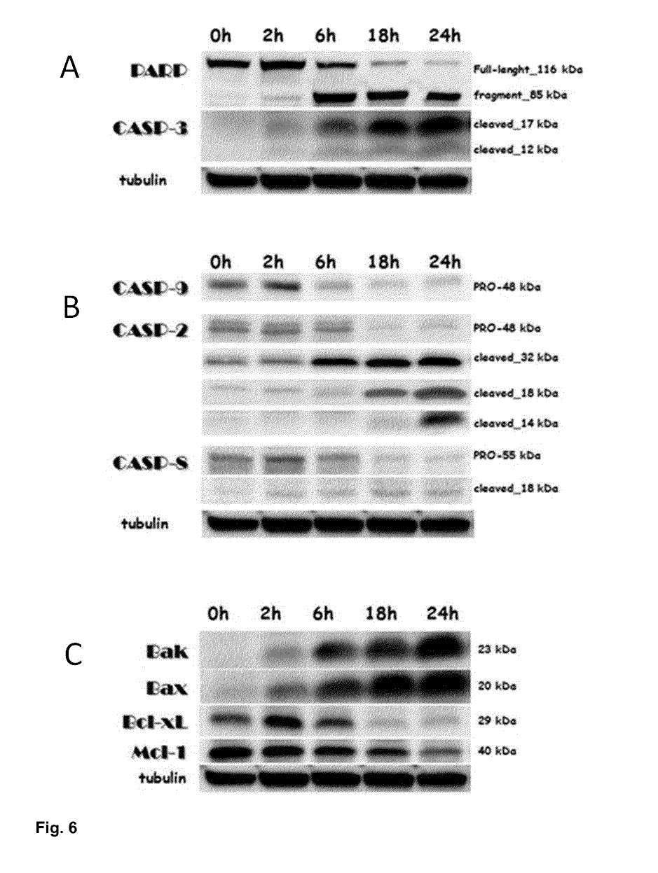

The antiproliferative effect of cannabinoids is mediated by apoptotic mechanisms. To evaluate which apoptotic signals could be involved in the anti-proliferative effect of cannabinoids on MM cells, we performed a time-course study by Western blot assays using the most resistant MM cell line, U266, upon exposure to WIN-55. We observed a decrease of full-length PARP, along with an increase of the 89 kDa fragment, which became detectable as early as 2 h after exposure to the drug (FIG. 6a). Next, we evaluated Casp-3 activation using an antibody that recognizes the active forms of Casp-3, this is, the 17 kDa- and 12 kDa-cleaved forms. We noticed that expression of the two active-cleaved forms of Casp-3 increased over time, being consistent with the increase of the fragmentation of PARP.

It is known that Casp-3 activation occurs by upstream caspases, such Casp-9, Casp-8 and Casp-2, and that they are initiator caspases that trigger intrinsic, extrinsic and endoplasmic reticulum-stress pathways, respectively. To know which of the above apoptotic signaling pathways was activated by cannabinoid treatment, we analyze the activity of their expression levels over time. As shown FIG. 6b, we detected a decrease of pro-Casp-9 after 2 h of exposure to cannabinoid and this processing moderately increased over time. Similarly, the level of full-length Casp-8 also began to fall after 2 h of exposure, but this decrease was slightly lower. We also detected the 18 kDa-cleaved form of Casp-8, which slightly appeared at 2 h and remained unchanged over time afterwards. The other active form of Casp-8, p10 fragment, was not detected at any time. Lastly, the processing of Casp-2 was evidenced by a decrease in the pro-caspase form and a temporally correlated increase of 32 kDa-, 18 kDa- and even of 12 kDa-cleaved forms, which are functionally active. Noteworthy, Casp-2 was fully processed, which is in contrast to Casp-8 and Casp-9. In addition the cleavage of Casp-2 was much more remarkable, particularly the p18 fragment, than that observed for caspase-8 and -9.

To further elucidate the molecular mechanisms involved in the cannabinoid-induced apoptotis, we next analyzed several molecules of Bcl-2 family, such as Mcl-1, Bcl-xL, Bax and Bak. Western blot analysis (FIG. 6c) showed a remarkable increase of pro-apototic regulators Bak and Bax, slightly higher for Bax, while expression level of anti-apoptotic proteins, Bcl-xL and Mcl-1, decreased over time.

Together, these data indicate that the effect of cannabinoid on myelomatous cells is mediated, at least in part, by the upregulation of pro-apoptotic proteins and downregulation of anti-apoptotic ones. The intrinsic, extrinsic and Casp-2 apoptotic pathways were involved in cannabinoid-induced apoptosis, being that Casp-2 pathway the more strongly activated.

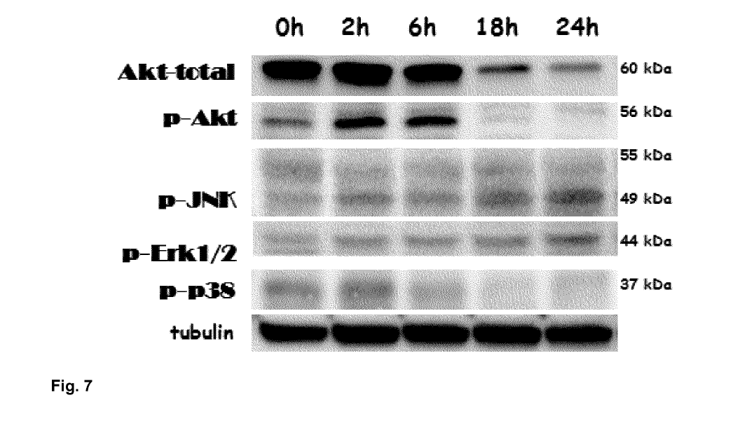

Signaling pathways targeted by cannabinoids in myelomatous cells. To get further insights into the mechanisms of cannabinoids-induced apoptosis, we assessed the expression profile of phosphorylated-JNK, -Erk1/2, -p38 MAPK and -Akt by Western blot. As shown FIG. 7 cannabinoid-treatment slightly up-regulated p-JNK and, to a lesser extent, p-Erk1/2 whereas moderately downregulated p-p38 MAPK in a time-dependent manner (FIG. 7). Intriguingly, WIN-55 induced a significant up-regulation of p-Akt at early time points, while at later times the expression levels exhibited a sharp decline. In contrast to p-Akt, total Akt-expression remained unchanged along the first 6 hours of exposure to the drug. However, after 6 h of incubation also total Akt exhibited a similar decrease as p-Akt (FIG. 7).

Together, these findings suggest that the effect of cannabinoids on myelomatous cells implicate different signaling pathways involved in survival, proliferation and cell death. Regarding Akt pathway, our results reveal a biphasic response, consisting of a short-term activation and a long-term downregulation.

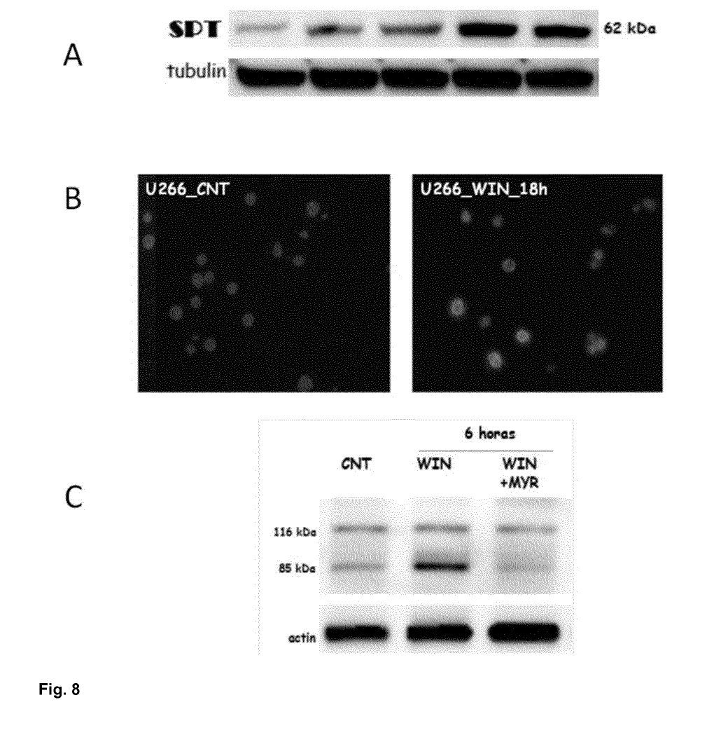

De novo synthesis of ceramides contribute to the apoptotic process triggered by cannabinoids. Next we examined whether ceramides participate in the cannabinoid-induced apoptosis in myelomatous cells. To this end, we analyzed the expression of serine palmitoyltransferase (SPT), the enzyme that catalyses the rate-limiting step of ceramide synthesis de novo. Our results show that SPT expression level in myelomatous cells is enhanced upon incubation with WIN-55 (FIG. 8a). As early as 2 h after exposure to the drug, we observed a moderate increase of SPT, which reached the maximal level at 18 h. To ensure that this up-regulation of SPT entails an increased of ceramide synthesis, we examined the expression of ceramide by immunohistochemistry. As shown in FIG. 8b, a remarkable increase of ceramide was detected in cannabinoid-treated U266 cells as compared to untreated cells. Furthermore, to strengthen the engagement of the ceramide in cannabinoid-induced apoptosis, we analyzed the cannabinoid-induce apoptosis after inhibition of ceramide synthesis with Myoricin, a selective SPT inhibitor. We observed that the pharmacological blockage of ceramide synthesis abolished, at least in part, the fragmentation of PARP induced by cannabinoid (FIG. 8c).

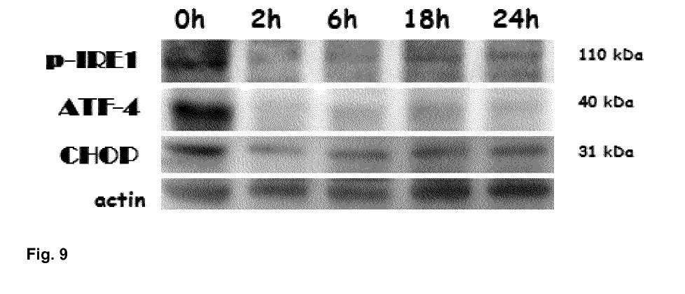

Cannabinoids attenuate the response to ER-stress in myelomatous cells. Due to the production of high levels of monoclonal immunoglobulins, myelomatous cells display a remarkably developed ER, so that they are prone to ER-stress. To test the effect of cannabinoids on ER-stress we analyzed the expression profile of ER-stress related proteins in myelomatous cells. As shown in FIG. 9, a slight and sustained decrease expression of CHOP, ATF-4 neither p-IRE1 was observed in cannabinoid-treated cells as compared to control cells. These results indicate that cannabionds decreased the unfolded-protein response in myelomatous cells.

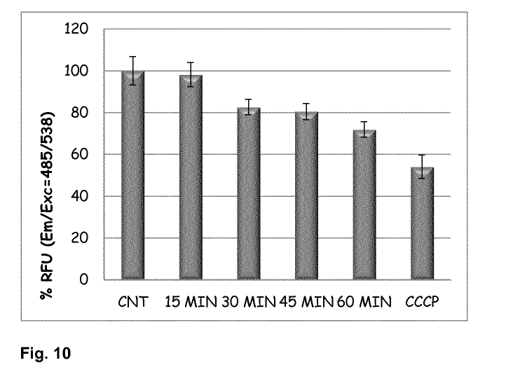

The early loss of mitochondrial-membrane potential induced by cannabinoids. To delve into the processes that participate in cannabinoid-induced apoptosis, finally we tested whether the cannabinoid treatment induces changes on Δψm in myelomatous cells. We observed that cannabinoids induced a drop of Δψm in U266 cells (FIG. 10). The loss of potential started as early as 15 min after exposure to the cannabinoid, and continued decreasing over time.

### Antiproliferative Effect in MM (In Vivo Assays)

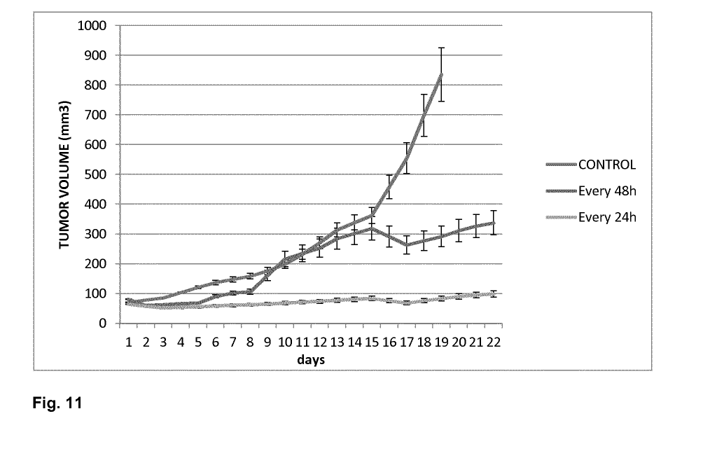

The administration with cannabinoids inhibits remarkably the growth of tumor in vivo. Finally, we analyzed the anti-tumor effect of the cannabinoids in vivo, using a model of human MM xenografted in immunodeficient mice NOD/SCID. We observed a remarkable and progressive loss of tumor-volume following cannabinoid administration as compared to vehicle-treated counterparts (FIG. 11). Our results, not only confirmed the above mentioned in vitro studies, but showed that cannabinoid administration not only slowed tumor growth but even reversed tumors in plasmacytoma-bearing mice.

### Discussion

Since the identification of the endocannabiniod system, the cannabinoid research on the oncology field has mainly focus on its antiemetic activity, whereas their antineoplasic properties were not addressed until late 90s in studies conducted in glioma (35) and breast cancer (36) cell lines. Now we report for the first time on the anti-tumor potential of cannabinoids for the treatment of multiple myeloma. We observed a remarkable decrease in myelomatous cells viability, both cell lines and primary plasma cells from myeloma patients, upon exposure to cannabinoids. By contrast, cannabinoids had no effect on normal healthy cells, including hematopoietic progenitor stem cells. Accordingly, cannabinoids exhibit a very selective anti-myeloma effect. Several studies had suggested that the expression level of CBs is correlated with susceptibility to cannabinoids and/or degree of malignancy (34, 35). According our results, the heterogeneity of the CBs pattern in hematopoietic cells is far too complex to conclude a clear relationship between receptor expression and sensitivity to the drug, so that further characterization of the entire profile is required to infer any correlation.

The analysis of PARP and Caspases reveals that cannabinoids trigger the two classical apoptotic signaling pathways, extrinsic and intrinsic, as well as the Casp-2-triggered pathway. The processing of Casp-2, -8 and -9 are observed as early as 2 h after exposure to the drug, but the activation over time of Casp-2 is notably stronger and, moreover, the up-regulation of cleaved forms of Casp-2 is parallel to the up-regulation of the PARP-fragment, which suggests a dominant role of Casp-2. In this regard, increasing evidence suggests that Casp-2 can function as initiator of apoptosis (37-38; Shi, 2005; Lava et al., 2012), It has been shown that involvement of Casp-2 occurs upstream of both mitochondrial damage and Casp-3 activation and, under stress conditions, a rapid up-regulation of Casp-2 occurs before induction of apoptosis (43). In multiple myeloma it has also been described that its activation occurs upstream of Casp-9 upon treatment with bortezomib (42). In accordance with these studies, our results suggest that Casp-2 plays a central role in the cannabinoid-induced apoptosis. Remarkably, Casp-2 is localized into ER membrane (43) and early studies have shown that it can be activated through ER-stress (42-43; 45-46). The ER responds to the burden of unfolded proteins (ER stress) by activating the unfolded protein response (UPR). UPR activation increases ER abundance to match needs by mediating expansion of the ER membrane. As such, the UPR establishes and maintains homeostasis (Peter Walter and David Ron. The Unfolded Protein Response: From Stress Pathway to Homeostatic Regulation Science 334, 1081 (2011). Due to the production of high levels of monoclonal immunoglobulins, myelomatous cells present a highly-developed ER and this makes MM cells particularly vulnerable to perturbations on protein metabolism (White-Gilbertson et al., 2013). In fact, the ER from myelomatous cells was referred as their “Achilles heel” (Pelletier et al., 2006), (Oslowski and Urano, 2011).

Therefore, to know the involvement of ER-stress in cannabinoid-induced apoptosis in myelomatous cells, we explored the expression profile of different proteins related to UPR before and after exposure to the drugs. Remarkably, myeloma cells lines display a high expression levels of UPR proteins, including CHOP, p-IRE1 and ATF-4, and this expression decreased upon exposure to cannabinoids. This result is in contrast to our own experience using leukemic cell cell lines (unpublished data) and also to the experience reported by other authors using glioma cell lines. It could be speculated that either cannabinoids attenuate ER stress or, on the contrary, impair the ability of MM cells to use the cytoprotective role of the UPR (Carrasco et al., Cancer Cell 11, 349 (2007; I. Papandreou et al., Blood 117, 1311 (2011). Finally, it is worth keeping in mind that UPR may provide opposing signals between cytoprotection and apoptosis.

Remarkably, RE is involved in the novo synthesis of ceramides (47, 48) which play a major regulatory role in apoptosis, enhancing the signaling events that drive apoptosis (49-52). In the current study we found that cannabinoids upregulate SPT and increase the levels of ceramides, as confirmed by immunohistochemistry assays. In addition, the inhibition of SPT by myriocin abolished fragmentation of PARP, thus confirming the central role of ceramide synthesis in the pro-apoptotic effect of cannabinoids. Our results are consistent with previous studies conducted in glioma cells showing that cannabinoids may induce accumulation of ceramide mainly by de novo synthesis (18, 27-29, 53).

It is worth mentioning the early loss of potential of mitochondrial-membrane, which occurs even before the cleavage of caspases. A previous study describes that the permeabilization of mitochondria can occur before and independently of caspase-3 activation (54), although active caspase-3 is required for the sustained loss of the mitochondrial transmembrane potential. In line with our observation, there are also evidences suggesting that sufficient amounts of ceramides can move from RE to mitochondria permeabilizing the mitochondrial membrane very early after drug exposure, even before caspases activation (47, 55). This early permeabilization of mitochondria could also be in accordance with the predominant role of Casp-2, since it can directly interact with the outer mitochondria membrane disrupting organelle function and triggering the release of proteins (38, 56).

In addition, several regulator factors of BCL-2 family, such Bax and Bak, actively participe in the permeabilization of mitochondrial membrane (57, 58) forming pores in the membrane by oligomerization and favouring mitochondrial outer membrane permeabilization (MOMP) during the induction phase of apoptosis (59). We found that cannabinoids remarkably upregulated the levels of both Bax and Bak at early time-points, which is consistent with the early loss of mitochondrial potential. Furthermore, it is known that channels formed by ceramides play a crucial role in the mitochondrial permeabilization (55; 60, 61). Recent studies show that Bak is required to drive the development of ceramide-channels (62). In turn, ceramide forms a platform into which Bax inserts, stabilizating the ceramide channels (63, 64). Moreover, along with this upregulation of Bax and Bak, we observed a remarkable downregulation of Bcl-xL and Mcl-1 induced by cannabinoids in MM cells. Interestingly, Bcl-xL also interacts with ceramide channels but exerts an opposite effect to Bax. In addition, it has been suggested that ceramide accumulation promotes inhibition of Mcl-1 and Bcl-xL (65, 66). Finally, is it worth mentioning that Mcl-1 and Bcl-xL are important for myelomatous cells survival (67-69) and an increased expression of both proteins has been associated to drug resistance (62, 69, 70). Overall, our results strength the notion that the ceramide channels and Bcl-2 proteins cooperate to promote early mitochondrial permeabilization induced by cannabinoids in MM cells.

Several signaling pathways have been reported that are affected by cannabinoids, such as mitogen-activated protein kinases (MAPKs) (26, 71-74) and P13K/Akt (PKB) (23, 27). In our study, we detected an unusual pattern of response of the AKT pathway to cannabinoids. Akt exhibits a biphasic pattern of expression after drug exposure. At short time-points, cannabinoids strongly activate Akt, but after 18 h the levels of phosphorylated Akt sharply decreases along with a remarkable down-regulation of Akt. This is in agreement to a previous study in glioma cells (75), describing an initial and acute rise of ceramide that stimulates Akt, while the sustained ceramide accumulation promotes Akt inhibition. Regarding MAPKs pathways, we found that cannabinoids promote a moderate activation of JNK, as well as inhibition of p38 signaling, whereas Erk pathway exhibits a slight activation at latter time points. The regulation of apoptosis by MAPKs is more complex than initially thought and often controversial. Thus, the functional significance in response to cannabinoids is difficult to interpret since these pathways crosstalk regulating each other. Nevertheless, given the low magnitude of regulation that we observed in the MAPKs pathways, we suggest that the involvement of MAPKs might be consequence of the early activation of ceramide/ER-stress/Casp-2 pathway, or secondary to the processes triggered during the apoptotic response. In agreement to this concept, a previous study suggested that ceramide can regulate p38-MAKP and JNK phosphorylation (76). Further studies are needed to discriminate the precise role of each kinase in the signaling cascade triggered by cannabinoids.

Finally, our study represents the first evidence of a successful in vivo treatment with cannabinoids in a murine xenograft model of multiple myeloma. U266 cell line, being the most resistant to cannabinoid-induced apoptosis in vitro, was selected to perform in vivo studies. Even so, we found that cannabinoids induce an impressive tumor growth inhibition, without mediating overt toxicity. Although conclusions from our in vivo study must be validated in MM patients, the results collectively suggest that a cannabinoids are a promising treatment for MM.

In summary, we report for the first time on the antitumor potential of cannabinoids for the treatment of multiple myeloma. Our study demonstrates a remarkable apoptotic effect of the cannabinoids WIN-55 and JWH-133 on myelomatous cells. Furthermore we observed that the apoptotic effect of cannabinoids is selective and do not affect the healthy counterpart cells. Cannabinoid-induced apoptosis involves the cooperation of different signaling mechanisms, highlighting the participation of ceramide and caspase-2. This study lays the groundwork for the design of new anti-myeloma therapies.

### Example 2

### Material and Methods

### Ethics Statement

This study was approved by the Ethics Committee for Clinical Research (CEIC) of the University Hospital Virgen del Rock:), and informed consents were obtained from all donors in accordance with the Declaration of Helsinki.

### Cell Cultures and Primary Cells

Human primary cells were obtained from LMA patients' bone marrow (BM) and healthy donors' peripheral blood (PB). Hematopoietic progenitor cells (CD34+) were isolated from leukaphesesis samples, and B (CD19+) and T (CD3+) lymphocytes from buffy coats by positive immunomagnetic separation in the AutoMACS pro separator (Miltenyi Biotec, Bergisch Gladbach, Germany). For this purpose CD34-, CD19- and CD3-MACS microbead Human Kit (Miltenyi Biotec, Bergisch Gladbach, Germany).

Human acute myeloid leukemia cell line KG-1 and human acute promyelocytic leukemia cell line HL60 (American Type Culture Collection) were cultured RPMI 1640 medium (Gibco™, Invitrogen, Barcelona, Spain) with 2 mM L-glutamine, 100 IU/m1 penicillin and 100 ug/ml streptomycin (all from Sigma-Aldrich, St. Louis, Mo., USA), and supplemented with 10% (HL60 cell line) or 20% (KG1 cell line and human primary cells) fetal bovine serum [(FBS), [Thermo Fisher Scientific (Waltham, Mass., USA)] in a humidified atmosphere of CO2/air (5%/95%).

### Drugs and Treatments

The cannabinoid agonists WIN-55,212-2 mesylate ((R)-(+)-[2,3-dihydro-5-methyl-3-(4-morpholinylmethyl)-pyrrolo-[1,2,3]-1,4-benzoxazin-6-yl]-1-naphthalenylmethanone mesylate) and JWH-133 ((6aR,10aR)-3-(1,1-Dimethylbutyl)-6a,7,10,10a-tetrahydro-6,6,9-trimethyl-6H-dibenzo [b,d] pyran) were purchased from Tocris Bioscience (Bristol, UK) and they was added in the indicated concentrations to culture medium for different incubation periods. Control cells were cultured with the relevant amounts of DMSO.

Myriocin (ISP-1), the inhibitor of the serin-palmitoyl-transferase (SPT) enzyme, was obtained from Enzo Life Sciences (Lausen, Switzerland). Thirty minutes before the experiment, the cells were transferred to serum free medium.

### Reagents and Antibodies

Antibodies for detection CB1- and CB2-receptor, caspases-2, -3, -8, -9, and phosphorilated forms of signaling pathways molecules Akt, Erk 1/2, p38MAPK, JNK and SPT were obtained from Abcam Company (Cambridge, UK). For Western Blot analyses, as a loading control were used anti-beta-actin and anti-beta-tubulin (Sigma-Aldrich). Antibodies against proteins PARP, Mcl-1, Bcl-xL, Bax and Bak were from BD Biosciences. All used secondary antibodies were Horseradish Peroxidase (HRP)-conjugated (Jackson ImmunoResearch Labs., Pa., USA), and produced in donkey to avoid potential cross-reactivity when multiple-probings were performed.

### Cell Viability MTT Assay

Cells lines, CD34+ cells and blast were cultured in 96-well plates (5×10e5 cells per well) with the addition of the indicated concentrations of WIN 55,212-2 or JWH-133 in DMSO or with the solvent alone in triplicate. Also we add myoricin together the cannabinoid WIN-55 to see if this effect reversed. Cell viability was determined by the WST-1 [2-(4-lodophenyl)-3-(4-nitrophenyl)-5-(2,4-disulfophenyl)-2H-tetrazolium] assay as per the manufacturer's instructions (Dojindo Molecular Technologies). Optical densities were measured at 450 nm using a plate reader Multiskan™ Go Microplate (Thermo Fisher Scientific, Waltham, Mass., USA).

Mitochondrial membrane potential and superoxide detection Cells (10e6 cells per assay) were treated with a concentration of WIN-55 for 15, 30, 45 and 60 min at 37° C. Fluorochromes 10 μM TMRM [(tetramethylrhodamine-ethyl-ester-perchlorate), Santa Cruz Biotechnology Inc.] to detect membrane potential, or 5 μM MitoSOX [Molecular Probes (Invitrogen)] to detect mitochondrial superoxide, were added for the last 15 min of treatment. CCCP (uncoupler of oxidative phosphorylation) or H₂O₂ (oxidant agent) was used as a positive control of the techniques respectively (Labbe et al., 2008). The fluorescence detection of TMRM was measured for flow citometry. Fluorescent intensity was quantified and analyzed using Infinicyt™ Software (Cytognos, Salamanca, Spain).

The MitoSOX signal was detected using a ‘Fluoroscan’ multi-well plate reader (Thermo Labsystems, Thermo Scientific), excitation/emission maxima of 543/593 nm.

### Protein Sample Preparation and Western Blot Analysis

Protein analyses were performed by Western blot in several sets of assays: expression pattern of CBs-receptors in AML cell lines and primary cells, and time course after treatment with cannabinoids in AML cell lines.

Cells were solubilized with ice-cold lysis buffer containing 2% ASB-14 (CalbioChem, San Diego, Calif., USA), 1% Nonidet P-40 (Ipegal®), 137 mM NaCl, 20 mM Tris-HCl pH 7.5 at 4° C. (all from Sigma-Aldrich), 2 mM Na₃VO₄, 20 mM NaF (New England BioLabs Inc., Ipswich, Mass., USA), 2 mM DTT (AppliChem, Darmstadt, Germany), protease inhibitor cocktail (Roche Diagnostics GmbH, Mannheim, Germany), and 10 ul Nuclease Mix (Amersham GE Healthcare, Uppsala, Sweden). Insoluble materials were removed by centrifugation at 13,000×g for 15 min at 4° C. Protein content was determined by Pierce® Microplate BCA Protein Assay kit-Reducing Agent Compatible (Thermo Fisher Scientific).

Extracted proteins (15-25 ug/well) were separated by sodium dodecyl sulfate-polyacrylamide gel electrophoresis (SDS/PAGE) in pre-cast polyacrylamide gels AnykD (Bio-Rad Laboratories, Hercules, Calif., USA) under reducing and denaturing conditions. Electrophoretic run was performed at a constant current of 15 mA per gel in a Mini-Protean® Tetra Cell (Bio-Rad Laboratories) and were electrophoretically transferred onto PVDF membranes using Trans-Blot® Turbo™ System (Bio-Rad Laboratories). Blocking was performed with 2% bovine serum albumin [(BSA), Santa Cruz Biotechnology] in Tween-Tris buffer saline (TTBS). Membranes were incubated overnight at 4° C. with corresponding primary antibody in TTBS.

Detection was performed using Western Blotting Luminol Reagent (Santa Cruz Biotechnology).

### Immunocytofluorescence Analysis of Culture Cells

Cells were grown on glass cover slips (Menzel-Glaser, Thermo Fisher Scientific) in the presence or absence of WIN 55,212-2. The cover slips were then washed twice in phosphate-buffered saline (PBS), and the cells were fixed in cold 4% paraformaldehyde for 10 min, treated with sodium borohydride and, permeabilized in 0.02% Triton X-100 in PBS with 10% normal donkey serum for blocking. Cell nuclei were stained with diamidino-2-phenylindole [(DAPI), Pierce, Thermo Fisher Scientific)]. Then, cells were incubated with the anti-CB1 or anti-CB2 receptor antibodies or anti-ceramide in blocking buffer overnight at 4° C. in a humid chamber (Simport, Beloeil, QC, Canada). After washing with PBS, samples were further incubated for 90 min with Alexa 647- or Alexa 488-conjugated donkey secondary antibody.

After final washing in PBS, the cover slips were dried and mounted on slides using Fluoromount Aqueous Mounting Medium (Sigma-Aldrich). Changes in the protein expression were visualized by fluorescence microscopy using a specific excitation for each antibody. Digital images were acquired with CellSens Dimension Software (Olympus) and manipulated with Photoshop CS2 Software (Adobe Systems Inc). There was no labeling when the primary antibody was omitted (data not shown).

### AML Murine Model

NOD/scid/IL-2R gammae null (NSG) mice were purchased from Charles River Laboratories International (L'Arbresle, France) and maintained with food and water ad libitum, under specific pathogen-free conditions. When mice were 8-12 weeks old, AML is induced by intratibial inoculation of the HL60 cell line. After being anesthetized, 1×10e6 cells are injected into the intra-osseous part of the tibia and the mice are monitored to monitor the progression of the disease by studying the weight loss and the detection of human CD45+ cells in the mouse peripheral blood by flow citometry.

When the presence of the disease is confirmed, treatment is initiated with the cannabinoid WIN-55 at a dose of 5 mg/kg, administered intraperitoneally. The animals are randomized into 3 groups: one group received the cannabinoid every 24 hours, another group at 48 h and the third group received the vehicle (RPMI+DMSO). Postmortem study is performed by flow cytometry to assess the effect of treatment of leukemia cells tested.

### Statistical Analyses

For all statistical analysis, SPSS software version 15.0 (Statistical Package for the Social Sciences, SPSS, Chicago, Ill., USA) was used and statistical significance was defined as P≤0.05. Error bars represent standard error of the mean (SEM). Data were analyzed using t-Student test.

### Results

### Antiproliferative Effect in AML (In Vitro Assays)

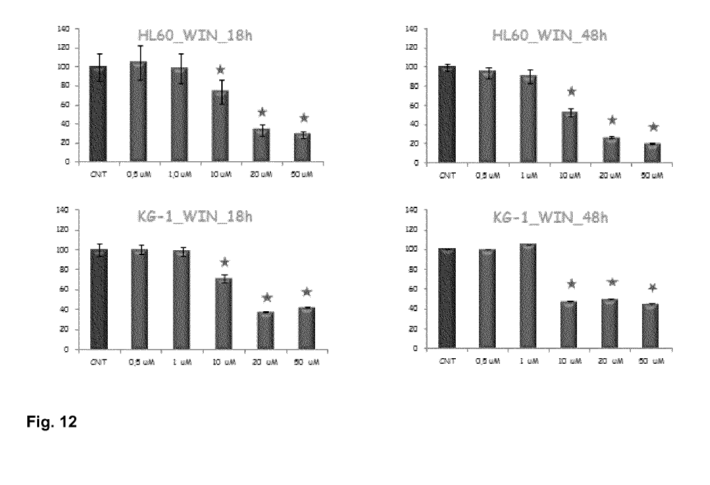

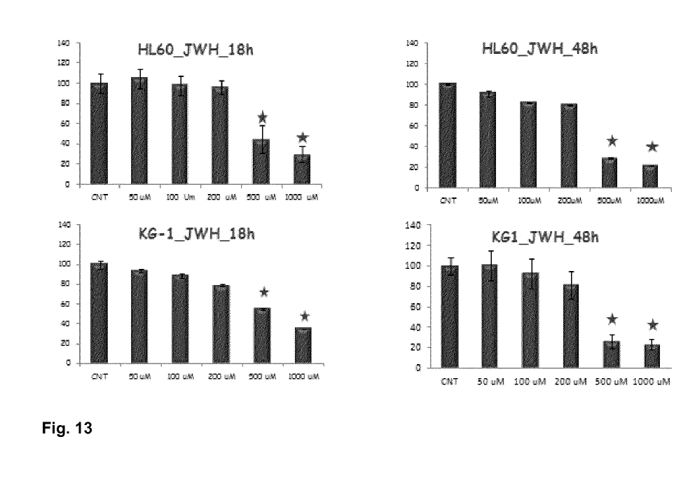

Cannabinoids have a highly selective antiproliferative effect in AML. Firstly, we assessed the effect of cannabinoids WIN-55 and JWH-133 on cell viability. To this end, the cells were treated with increasing concentrations of cannabinoids, WIN-55 (0.5-50 uM) and JWH-133 (0.05-1 mM), for 18 and 48, and viability was analyzed by MTT assays. We found that incubation with WIN-55 and JWH-133 for 18 h significantly reduced the cell viability of KG1 (acute myelogenous leukemia) and HL60 (Human promyelocytic leukemia cells) cell lines tested as compared to untreated control cells (FIG. 12 and FIG. 13). Although both cell lines were sensitive to treatment with WIN-55, HL60 was the most sensitive.

### Cell Viability

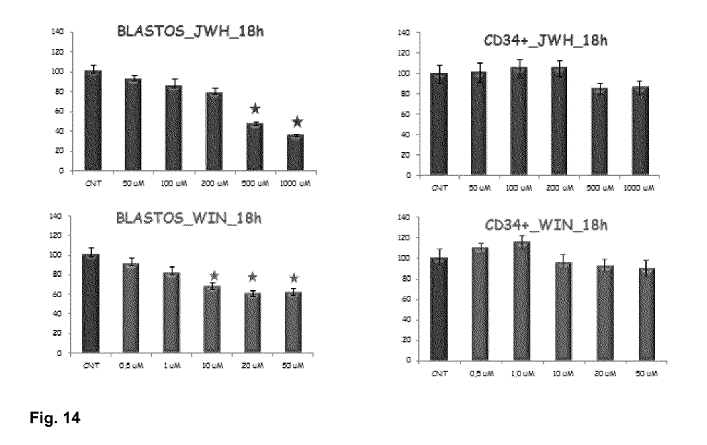

Cannabinoids reduce the cell viability of blasts from AML patients, but do not affect normal cell populations. Cell viability of CD34+ hematopoietic stem cells remains unaffected after cannabinoid treatment (FIG. 14). Stem cells were treated with WIN-55 or JWH-133 and cell viability was analyzed using MTT assay.

Taken together, our results indicate that cannabinoids, WIN-55 and JWH-33, have a very selective effect as they induce apoptosis on blasts from AML patients whereas viability of healthy cells, including CD34+ hematopoietic stem cells remained unaffected.

### Mechanism of Action

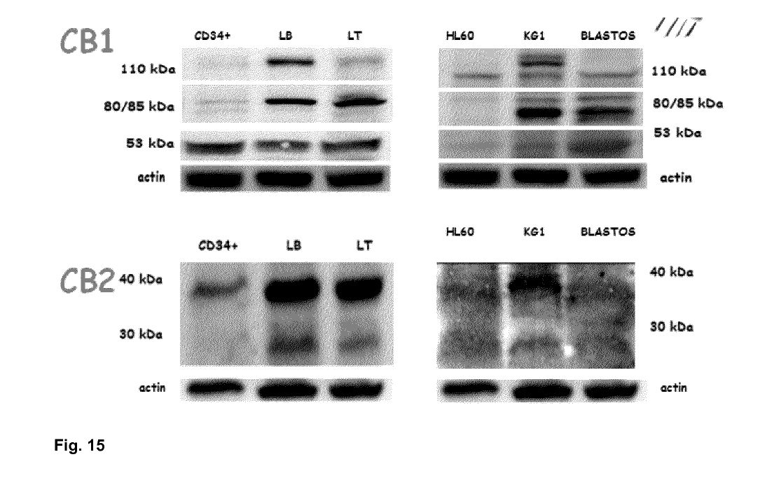

Profile of CBs receptors expression in AML cell lines and primary cells is very complex and heterogeneous. We analyzed by Western blot the expression pattern of cannabinoid receptors in primary cells from healthy donors and AML cell lines. We detected CB1 and CB2 expression in all cell subsets analyzed. Of note, both receptors showed multiple bands and a very heterogeneous pattern between the two cell subtypes analyzed (FIG. 15).

As shown in the case of MM cells, our results show that the expression of CBs, both CB1 and CB2 in AML cells is complex and more heterogeneous than previously suspected.

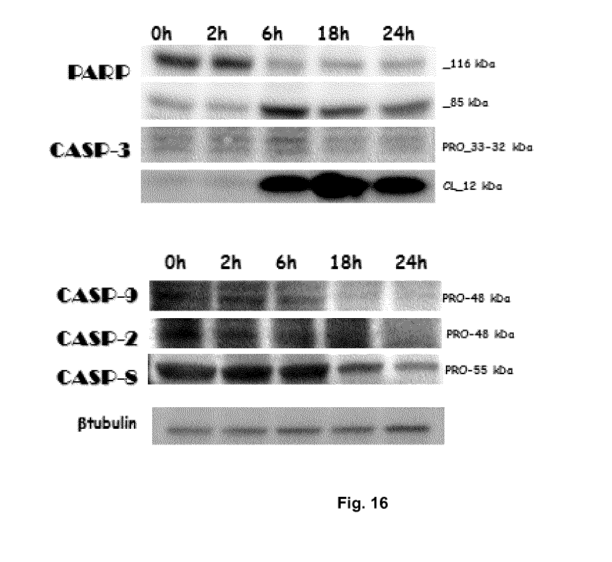

As in the case of MM cells, the antiproliferative effect of cannabinoids in AML is mediated by apoptotic mechanisms. To evaluate which apoptotic signals could be involved in the anti-proliferative effect of cannabinoids on AML cells, we performed a time-course study by Western blot assays using HL60 cells, upon exposure to WIN-55. We observed a decrease of full-length PARP, along with an increase of the 85 kDa fragment (FIG. 16a). Next, we evaluated Casp-3 activation using an antibody that recognizes the active forms of Casp-3, this is, the pro-Casp-3 and 12 kDa-cleaved forms. We noticed that expression of the active 12 kDa-cleaved form of Casp-3 increased over time, being consistent with the increase of the fragmentation of PARP.

It is known that Casp-3 activation occurs by upstream caspases, such Casp-9, Casp-8 and Casp-2, and that they are initiator caspases that trigger intrinsic, extrinsic and endoplasmic reticulum-stress pathways, respectively. To know which of the above apoptotic signaling pathways was activated by cannabinoid treatment, we analyze the activity of their expression levels over time. As shown in FIG. 16b, we detected a decrease of pro-Casp-9 after 2 h of exposure to cannabinoid and this processing moderately increased over time. Similarly, the level of full-length Casp-8 also began to fall after 2 h of exposure, but this decrease was slightly lower. Lastly, the processing of Casp-2 was evidenced by a decrease in the pro-caspase form which is functionally active.

Together, these data indicate that the effect of cannabinoid on leukemic cells is mediated, at least in part, by the upregulation of pro-apoptotic proteins and downregulation of anti-apoptotic ones.

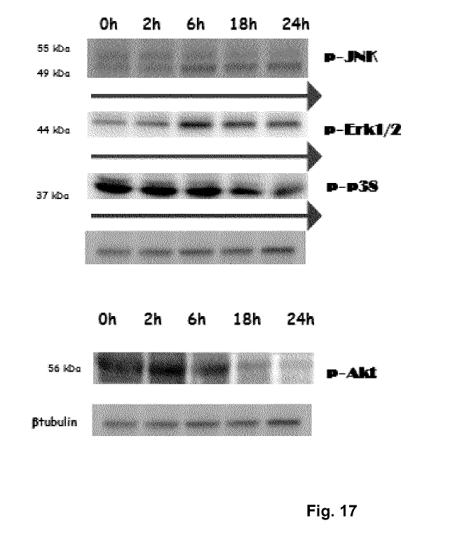

Signaling pathways targeted by cannabinoids in leukemic cells. To get further insights into the mechanisms of cannabinoids-induced apoptosis, we assessed the expression profile of phosphorylated-JNK, -Erk1/2, -p38 MAPK and -Akt by Western blot. As shown FIG. 17 cannabinoid-treatment slightly up-regulated p-Erk1/2 and, to a lesser extent, p-JNK, whereas significantly downregulated p-Akt and moderately downregulated p-p38 MAPK in a time-dependent manner. Together, these findings suggest that the effect of cannabinoids leukemic cells implicate different signaling pathways involved in survival, proliferation and cell death being the Akt/PKB pathway the main transduction signal triggered by WIN-55.

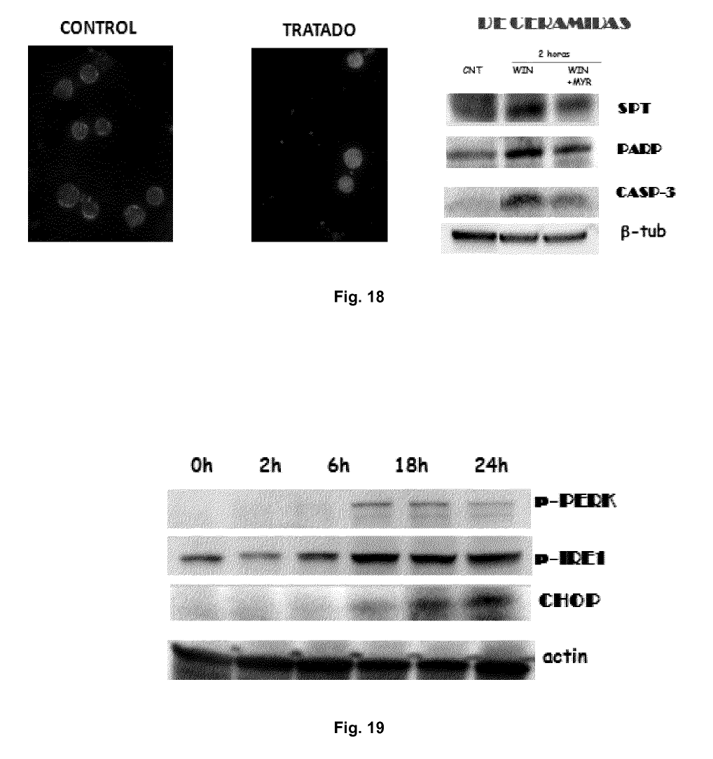

Additionally, de novo synthesis of ceramides also contribute to the apoptotic process triggered by cannabinoids in AML. We examined whether ceramides participate in the cannabinoid-induced apoptosis in leukemic cells. To this end, we analyzed the expression of serine palmitoyltransferase (SPT), the enzyme that catalyses the rate-limiting step of ceramide synthesis de novo. Our results show that SPT expression level in leukemic cells is enhanced upon incubation with WIN-55 (FIG. 18). As early as 2 h after exposure to the drug, we observed a moderate increase of SPT. To ensure that this up-regulation of SPT entails an increased of ceramide synthesis, we examined the expression of ceramide by immunohistochemistry. As shown in FIG. 18, a remarkable increase of ceramide was detected in cannabinoid-treated HL60 cells as compared to untreated cells. Furthermore, to strengthen the engagement of the ceramide in cannabinoid-induced apoptosis, we analyzed the cannabinoid-induce apoptosis after inhibition of ceramide synthesis with Myoricin, a selective SPT inhibitor. We observed that the pharmacological blockage of ceramide synthesis abolished, at least in part, the fragmentation of PARP induced by cannabinoid (FIG. 18).

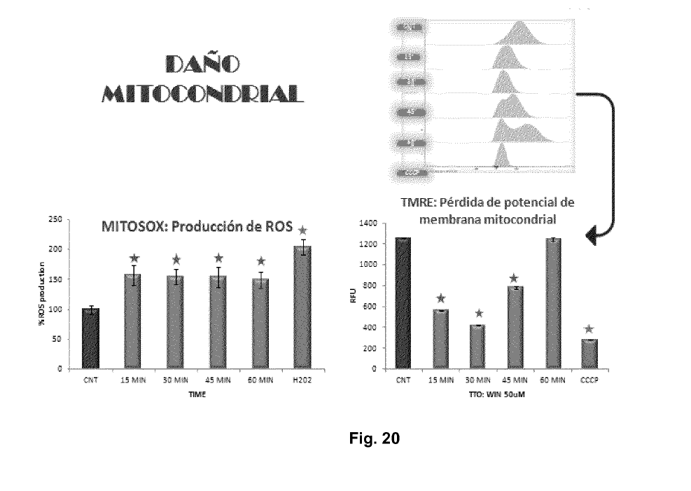

Cannabinoid treatment promotes an early loss of potential of mitochondrial membrane. To delve into the processes that participate in cannabinoid-induced apoptosis, we evaluated the potential of mitochondrial membrane potential in HL60 cells after treatment with WIN-55, at indicated times, using TMRE assay by flow citometry. We observed that cannabinoids induced a drop of Δψm in HL60 cells (FIG. 20a). The loss of potential started as early as 15 min after exposure to the cannabinoid, and continued decreasing over time. Also the ROS increase by MitoSOX assay was observed, specially in HL60 cells after exposure to WIN-55 (FIG. 20b).

### Antiproliferative Effect in AML (In Vivo Assays)

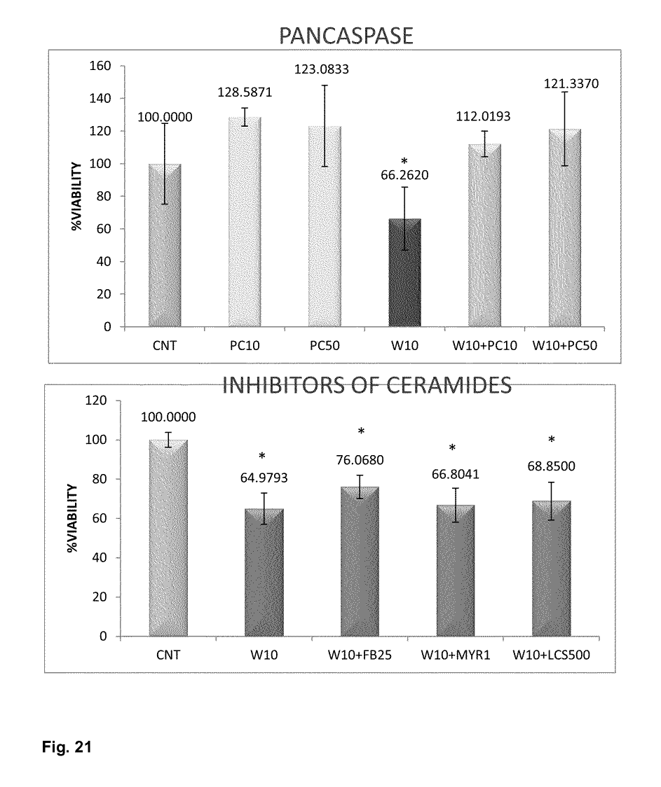

The administration with cannabinoids inhibits remarkably the growth of tumor in vivo. Finally, we analyzed the anti-tumor effect of the cannabinoids in vivo, using a model of human AML (HL60) xenografted in immunodeficient mice NOD/SCID. We observed a significant increase in the mice survival following cannabinoid administration as compared to vehicle-treated counterparts (FIG. 21).

### REFERENCES

1. Anderson K C, Carrasco R D. Pathogenesis of myeloma. Annu Rev Pathol. 2011; 6:249-274.
2. Munshi N C, Anderson K C. Minimal residual disease in multiple myeloma. J Clin Oncol. 2013; 31(20):2523-6.
3. Mackie K. Cannabinoid receptors: where they are and what they do. J Neuroendocrinol. 2008; 20 Suppl 1:10-4.
4. Onaivi E S. An endocannabinoid hypothesis of drug reward and drug addiction. Ann N Y Acad Sci. 2008; 1139:412-21.
5. Matsuda L A, Lolait S J, Brownstein M J, Young A C, Bonner T I. Structure of a cannabinoid receptor and functional expression of the cloned cDNA. Nature. 1990; 346(6284):561-4.
6. Munro S, Thomas K L, Abu-Shaar M. Molecular characterization of a peripheral receptor for cannabinoids. Nature. 1993; 365(6441):61-5.
7. Galiegue S, Mary S, Marchand J, Dussossoy D, Carrière D, Carayon P, Bouaboula M, Shire D, Le Fur G, Casellas P. Expression of central and peripheral cannabinoid receptors in human immune tissues and leukocyte subpopulations. Eur J Biochem. 1995; 232(1):54-61.
8. Howlett A C, Barth F, Bonner T I, Cabral G, Casellas P, Devane W A, Felder C C, Herkenham M, Mackie K, Martin B R, Mechoulam R, Pertwee R G. International Union of Pharmacology. XXVII. Classification of cannabinoid receptors. Pharmacol Rev. 2002; 54(2):161-202.
9. Di Marzo V1, De Petrocellis L, Fezza F, Ligresti A, Bisogno T. Anandamide receptors. Prostaglandins Leukot Essent Fatty Acids. 2002; 66(2-3):377-91.
10. De Petrocellis L, Di Marzo V. An introduction to the endocannabinoid system: from the early to the latest concepts. Best Pract Res Clin Endocrinol Metab. 2009; 23(1):1-15.
11. Mechoulam R, Panikashvili D, Shohami E. Cannabinoids and brain injury: therapeutic implications. Trends Mol Med. 2002; 8(2):58-61
12. Di lorio G, Lupi M, Sarchione F, Matarazzo I, Santacroce R, Petruccelli F, Martinotti G, Di

Giannantonio M. The endocannabinoid system: a putative role in neurodegenerative diseases. Int J High Risk Behav Addict. 2013; 2(3):100-6.
13. Davis M P. Cannabinoids in pain management: CB1, CB2 and non-classic receptor ligands. Expert Opin Investig Drugs. 2014; 23(8):1123-40.
14. Parker L A, Rock E M, Limebeer C L. Regulation of nausea and vomiting by cannabinoids. Br J Pharmacol. 2011; 163(7):1411-22.
15. Todaro B. Cannabinoids in the treatment of chemotherapy-induced nausea and vomiting. J Natl Compr Canc Netw. 2012; 10(4):487-92.
16. Bifulco M1, Laezza C, Pisanti S, Gazzerro P. Cannabinoids and cancer: pros and cons of an antitumour strategy. Br J Pharmacol. 2006; 148(2):123-35.
17. Rocha FC1, Dos Santos Júnior J G, Stefano S C, da Silveira D X. Systematic review of the literature on clinical and experimental trials on the antitumor effects of cannabinoids in gliomas. J Neurooncol. 2014; 116(1):11-24.
18. Galve-Roperh I, Sánchez C, Cortés M L, Gómez del Pulgar T, lzquierdo M, Guzmán M. Anti-tumoral action of cannabinoids: involvement of sustained ceramide accumulation and extracellular signal-regulated kinase activation. Nat Med. 2000; 6(3):313-9.
19. Sanchez C, de Ceballos M L, Gomez del Pulgar T, Rueda D, Corbacho C, Velasco G, Galve-Roperh I, Huffman J W, Ramón y Cajal S, Guzmán M. Inhibition of glioma growth in vivo by selective activation of the CB(2) cannabinoid receptor. Cancer Res. 2001; 61(15):5784-9.
20. Massi P, Vaccani A, Ceruti S, Colombo A, Abbracchio M P, Parolaro D. Antitumor effects of cannabidiol, a nonpsychoactive cannabinoid, on human glioma cell lines. J Pharmacol Exp Ther. 2004; 308(3):838-45.
21. Guzmán M, Duarte M J, Blázquez C, Ravina J, Rosa M C, Galve-Roperh I, Sánchez C, Velasco G, González-Feria L. A pilot clinical study of Delta9-tetrahydrocannabinol in patients with recurrent glioblastoma multiforme. Br J Cancer. 2006; 95(2):197-203.
22. Velasco G, Carracedo A, Blázquez C, Lorente M, Aguado T, Haro A, Sánchez C, Galve-Roperh I, Guzmán M. Cannabinoids and gliomas. Mol Neurobiol. 2007; 36(1):60-7.
23. Ellert-Miklaszewska A, Kaminska B, Konarska L. Cannabinoids down-regulate PI3K/Akt and Erk signalling pathways and activate proapoptotic function of Bad protein. Cell Signal. 2005; 17(1):25-37.
24. Rueda D, Galve-Roperh I, Haro A, Guzmán M. The CB(1) cannabinoid receptor is coupled to the activation of c-Jun N-terminal kinase. Mol Pharmacol. 2000; 58(4):814-20.
25. Herrera B, Carracedo A, Diez-Zaera M, Guzmán M, Velasco G. p38 MAPK is involved in CB2 receptor-induced apoptosis of human leukaemia cells. FEBS Lett. 2005; 579(22):5084-8.
26. Gómez del Pulgar T, Velasco G, Guzmán M. The CB1 cannabinoid receptor is coupled to the activation of protein kinase B/Akt. Biochem J. 2000; 347(Pt 2):369-73.
27. Gómez Del Pulgar T, De Ceballos M L, Guzmán M, Velasco G. Cannabinoids protect astrocytes from ceramide-induced apoptosis through the phosphatidylinositol 3-kinase/protein kinase B pathway. J Biol Chem. 2002; 277(39):36527-33.
28. Velasco G, Galve-Roperh I, Sanchez C, Blázquez C, Haro A, Guzmán M. Cannabinoids and ceramide: two lipids acting hand-by-hand. Life Sci. 2005; 77(14):1723-31.
29. Herrera B, Carracedo A, Diez-Zaera M, Gómez del Pulgar T, Guzmán M, Velasco G. The CB2 cannabinoid receptor signals apoptosis via ceramide-dependent activation of the mitochondrial intrinsic pathway. Exp Cell Res. 2006; 312(11):2121-31.
30. Bouaboula M, Rinaldi M, Carayon P, Carillon C, Delpech B, Shire D, Le Fur G, Casellas P. Cannabinoid-receptor expression in human leukocytes. Eur J Biochem. 1993; 214(1):173-80.
31. Van Sickle M D, Duncan M, Kingsley P J, Mouihate A, Urbani P, Mackie K, Stella N, Makriyannis A, Piomelli D, Davison J S, Marnett L J, Di Marzo V, Pittman Q J, Patel K D, Sharkey K A. Identification and functional characterization of brainstem cannabinoid CB2 receptors. Science. 2005; 310(5746):329-32.
32. Gong J P, Onaivi E S, Ishiguro H, Liu Q R, Tagliaferro P A, Brusco A, Uhl G R. Cannabinoid CB2 receptors: immunohistochemical localization in rat brain. Brain Res. 2006; 1071(1):10-23.
33. Onaivi E S, Ishiguro H, Gong J P, Patel S, Perchuk A, Meozzi P A, Myers L, Mora Z, Tagliaferro P, Gardner E, Brusco A, Akinshola B E, Liu Q R, Hope B, Iwasaki S, Arinami T, Teasenfitz L, Uhl G R. Discovery of the presence and functional expression of cannabinoid CB2 receptors in brain. Ann N Y Acad Sci. 2006; 1074:514-36.
34. Benito C, Tolón R M, Pazos M R, Núñez E, Castillo A I, Romero J. Cannabinoid CB2 receptors in human brain inflammation. Br J Pharmacol. 2008; 153(2):277-85.
35. Sánchez C, Galve-Roperh I, Canova C, Brachet P, Guzman M. Delta9-tetrahydrocannabinol induces apoptosis in C6 glioma cells. FEBS Lett. 1998; 436:6-10.
36. De Petrocellis L, Melck D, Palmisano A, Bisogno T, Laezza C, Bifulco M, Di Marzo V. The endogenous cannabinoid anandamide inhibits human breast cancer cell proliferation. Proc Natl Acad Sci U S A. 1998; 8375-80.
37. Guo Y, Srinivasula S M, Druilhe A, Fernandes-Alnemri T, Alnemri E S. Caspase-2 induces apoptosis by releasing proapoptotic proteins from mitochondria. J Biol Chem. 2002 Apr. 19; 277(16):13430-7
38. Gao Z, Shao Y, Jiang X. Essential roles of the Bcl-2 family of proteins in caspase-2-induced apoptosis. J Biol Chem. 2005 Nov. 18; 280(46):38271-5.
39. Krumschnabel G, Sohm B, Bock F, Manzl C, Villunger A. The enigma of caspase-2: the laymen's view. Cell Death Differ. 2009 February; 16(2):195-207.
40. Imre G, Heering J, Takeda A N, Husmann M, Thiede B, zu Heringdorf D M, Green D R, van der Goot FG, Sinha B, Dötsch V, Rajalingam K. Caspase-2 is an initiator caspase responsible for pore-forming toxin-mediated apoptosis. EMBO J. 2012 May 30; 31(11):2615-28
41. Bouchier-Hayes L, Oberst A, McStay G P, Connell S, Tait S W, Dillon C P, Flanagan J M, Beere H M, Green D R. Characterization of cytoplasmic caspase-2 activation by induced proximity. Mol Cell. 2009 Sep. 24; 35(6):830-40.
42. Gu H, Chen X, Gao G, Dong H. Caspase-2 functions upstream of mitochondria in endoplasmic reticulum stress-induced apoptosis by bortezomib in human myeloma cells. Mol Cancer Ther. 2008 August; 7(8):2298-307.
43. Upton J P, Austgen K, Nishino M, Coakley K M, Hagen A, Han D, Papa F R, Oakes S A. Caspase-2 cleavage of BID is a critical apoptotic signal downstream of endoplasmic reticulum stress. Mol Cell Biol. 2008 June; 28(12):3943-51.
44. Mishra R, Karande A A. Endoplasmic reticulum stress-mediated activation of p38 MAPK,

Caspase-2 and Caspase-8 leads to abrin-induced apoptosis. PLoS One. 2014 Mar. 24; 9(3):e92586.
45. Aksenova V I, Bylino O V, Zhivotovskii B D, Lavrik I N. Caspase-2: what do we know today? Mol Biol. 2013 March-April; 47(2):187-204.
46. Penna F, Pin F, Costamagna D, Reffo P, Baccino F M, Bonelli G, Costelli P. Caspase 2 activation and ER stress drive rapid Jurkat cell apoptosis by clofibrate. PLoS One. 2012; 7(9):e45327.
47. Stiban J, Caputo L, Colombini M. Ceramide synthesis in the endoplasmic reticulum can permeabilize mitochondria to proapoptotic proteins. J Lipid Res. 2008 March; 49(3):625-34.
48. Levy M, Futerman A H. Mammalian ceramide synthases. IUBMB Life. 2010 May; 62(5):347-56.
49. Hannun Y A. Sphingolipid second messengers: tumor suppressor lipids. Adv Exp Med Biol. 1997; 400A:305-12.
50. Siskind L J. Mitochondrial ceramide and the induction of apoptosis. J Bioenerg Biomembr. 2005 June; 37(3):143-53.
51. Mullen T D, Obeid L M. Ceramide and apoptosis: exploring the enigmatic connections between sphingolipid metabolism and programmed cell death. Anticancer Agents Med Chem. 2012 May; 12(4):340-63.
52. Morad S A, Cabot M C. Ceramide-orchestrated signalling in cancer cells. Nat Rev Cancer. 2013 January; 13(1):51-65.
53. Guzman M, Galve-Roperh I, Sanchez C. Ceramide: a new second messenger of cannabinoid action. Trends Pharmacol Sci. 2001 January; 22(1):19-22.
54. Waterhouse N J, Sedelies K A, Sutton V R, Pinkoski M J, Thia K Y, Johnstone R, Bird P I, Green D R, Trapani J A. Functional dissociation of DeltaPsim and cytochrome c release defines the contribution of mitochondria upstream of caspase activation during granzyme B-induced apoptosis. Cell Death Differ. 2006 April; 13(4):607-18.
55. Colombini M. Membrane channels formed by ceramide. Handb Exp Pharmacol. 2013; (215):109-26.
56. Robertson J D, Gogvadze V, Kropotov A, Vakifahmetoglu H, Zhivotovsky B, Orrenius S. Processed caspase-2 can induce mitochondria-mediated apoptosis independently of its enzymatic activity. EMBO Rep. 2004 June; 5(6):643-8.
57. Kuwana T, Mackey M R, Perkins G, Ellisman M H, Latterich M, Schneiter R, Green D R, Newmeyer D D. Bid, Bax, and lipids cooperate to form supramolecular openings in the outer mitochondrial membrane. Cell. 2002 Nov. 1; 111(3):331-42.
58. Letai A, Bassik M C, Walensky L D, Sorcinelli M D, Weiler S, Korsmeyer S J. Distinct BH3 domains either sensitize or activate mitochondrial apoptosis, serving as prototype cancer therapeutics. Cancer Cell. 2002 September; 2(3):183-92.
59. Westphal D, Dewson G, Menard M, Frederick P, Iyer S, Bartolo R, Gibson L, Czabotar P E, Smith B J, Adams J M, Kluck R M. Apoptotic pore formation is associated with in-plane insertion of Bak or Bax central helices into the mitochondrial outer membrane. Proc Natl Acad Sci U S A. 2014 Sep. 30; 111(39):E4076-85.
60. Perera M N, Lin S H, Peterson Y K, Bielawska A, Szulc Z M, Bittman R, Colombini M. Bax and Bcl-xL exert their regulation on different sites of the ceramide channel. Biochem J. 2012 Jul. 1; 445(1):81-91.
61. Siskind L J, Mullen T D, Romero Rosales K, Clarke C J, Hernandez-Corbacho M J, Edinger A L, Obeid L M. The BCL-2 protein BAK is required for long-chain ceramide generation during apoptosis. J Biol Chem. 2010 Apr. 16; 285(16):11818-26.
62. Beverly L J, Howell L A, Hernandez-Corbacho M, Casson L, Chipuk J E, Siskind L J. BAK activation is necessary and sufficient to drive ceramide synthase-dependent ceramide accumulation following inhibition of BCL2-like proteins. Biochem J. 2013 May 15; 452(1)111-9.
63. Lee H, Rotolo J A, Mesicek J, Penate-Medina T, Rimner A, Liao W C, Yin X, Ragupathi G, Ehleiter D, Gulbins E, Zhai D, Reed J C, Haimovitz-Friedman A, Fuks Z, Kolesnick R. Mitochondrial ceramide-rich macrodomains functionalize Bax upon irradiation. PLoS One. 2011; 6(6):e19783.
64. Chipuk J E, McStay G P, Bharti A, Kuwana T, Clarke C J, Siskind L J, Obeid L M, Green D R. Sphingolipid metabolism cooperates with BAK and BAX to promote the mitochondrial pathway of apoptosis. Cell. 2012 Mar. 2; 148(5):988-1000.
65. Bonhoure E, Lauret A, Barnes D J, Martin C, Malavaud B, Kohama T, Melo J V, Cuvillier O. Sphingosine kinase-1 is a downstream regulator of imatinib-induced apoptosis in chronic myeloid leukemia cells. Leukemia. 2008 May; 22(5):971-9.
66. Schubert K M, Duronio V. Distinct roles for extracellular-signal-regulated protein kinase (ERK) mitogen-activated protein kinases and phosphatidylinositol 3-kinase in the regulation of Mcl-1 synthesis. Biochem J. 2001 Jun. 1; 356(Pt 2):473-80.
67. Zhang B, Gojo I, Fenton R G. Myeloid cell factor-1 is a critical survival factor for multiple myeloma. Blood. 2002 Mar. 15; 99(6):1885-93.
68. Gomez-Bougie P, Wuillème-Toumi S, Ménoret E, Trichet V, Robillard N, Philippe M, Bataille R, Amiot M. Noxa up-regulation and Mcl-1 cleavage are associated to apoptosis induction by bortezomib in multiple myeloma. Cancer Res. 2007 Jun. 1; 67(11):5418-24.
69. Chen S, Dai Y, Pei XY, Myers J, Wang L, Kramer L B, Garnett M, Schwartz D M, Su F, Simmons G L, Richey J D, Larsen D G, Dent P, Orlowski R Z, Grant S. CDK inhibitors upregulate BH3-only proteins to sensitize human myeloma cells to BH3 mimetic therapies. Cancer Res. 2012 Aug. 15; 72(16):4225-37.
70. Yecies D, Carlson N E, Deng J, Letai A. Acquired resistance to ABT-737 in lymphoma cells that up-regulate MCL-1 and BFL-1. Blood. 2010 Apr. 22; 115(16):3304-13.
71. Liu J, Batkai S, Pacher P, Harvey-White J, Wagner J A, Cravatt B F, Gao B, Kunos G. Lipopolysaccharide induces anandamide synthesis in macrophages via CD14/MAPK/phosphoinositide 3-kinase/NF-kappaB independently of platelet-activating factor. J Biol Chem. 2003 Nov. 7; 278(45):45034-9.
72. Rueda D, Galve-Roperh I, Haro A, Guzmán M. The CB(1) cannabinoid receptor is coupled to the activation of c-Jun N-terminal kinase. Mol Pharmacol. 2000 October; 58(4):814-20.
73. Derkinderen P, Ledent C, Parmentier M, Girault J A. Cannabinoids activate p38 mitogen-activated protein kinases through CB1 receptors in hippocampus. J Neurochem. 2001 May; 77(3):957-60.
74. Bouaboula M, Poinot-Chazel C, Bourrié B, Canat X, Calandra B, Rinaldi-Carmona M, Le Fur G, Casellas P. Activation of mitogen-activated protein kinases by stimulation of the central cannabinoid receptor CB1. Biochem J. 1995 Dec. 1; 312 (Pt 2):637-41.
75. Guz\acute{m}an M. Cannabinoids: potential anticancer agents. Nat Rev Cancer. 2003 October; 3(10):745-55.
76. Chen C L, Lin C F, Chang W T, Huang W C, Teng C F, Lin Y S. Ceramide induces p38 MAPK and JNK activation through a mechanism involving a thioredoxin-interacting protein-mediated pathway. Blood. 2008 Apr. 15; 111(8):4365-74.
77. Gilbert C, Rollet-Labelle E, Caon A C, Naccache P H. Immunoblotting and sequential lysis protocols for the analysis of tyrosine phosphorylation-dependent signaling. J Immunol Methods. 2002; 271:185-201.

1. A ceramide-generating anticancer agent or treatment and/or a ceramide degradation inhibitor, or a pharmaceutically acceptable salt or ester thereof, for its use in the prevention, treatment, or amelioration of cancer or tumors of the hematopoietic and lymphoid tissues.

2. The ceramide-generating anticancer agent or treatment, and/or the ceramide degradation inhibitor according to claim 1, wherein is a cannabinoid.

3. The ceramide-generating anticancer agent or treatment, and/or the ceramide degradation inhibitor according to anyone of claims 1-2, wherein the cannabinoid is selected from the group consisting of: HU-308; JWH-133; L-759, 633; PRS 211,375 (Cannabinor); AM-1241; JWH-015; L-759, 656; GW-842, 166X; GP-1 a; THC (Tetrahidrocannabinol); HU-210; L-759, 656; WIN 55,212-2; CP 55940; CRA-13; SAB-378; JWH-018 (or AM-678); CP 50,556-1 (levonantradol), or combinations thereof.

4. The ceramide-generating anticancer agent or treatment, and/or the ceramide degradation inhibitor according to anyone of claims 1-3, wherein the cannabinoid is WIN 55,212-2 or a pharmaceutically acceptable salt or ester thereof.

5. The ceramide-generating anticancer agent or treatment, and/or the ceramide degradation inhibitor according to anyone of claims 1-3, wherein the cannabinoid is JWH-133 or a pharmaceutically acceptable salt or ester thereof.

6. The ceramide-generating anticancer agent or treatment, and/or the ceramide degradation inhibitor according to anyone of claims 1-5, wherein the cannabionid agent is natural and is selected from the group consisting of: cannabigerol-type agent (CBG), cannabichromene-type agent (CBC), cannabinodiol-type agents (CBND), tetrahydrocannabinol-type agents (THC), cannabinol-type agents (CBN), cannabitriol agents (CBT), cannabielsoin agent (CBE), isocannabinoids agents, cannabiciclol-type agents (CBL), cannabicitran-type agents (CBT), cannacichromanone-type agents (CBCN), or combinations thereof.

7. The ceramide-generating anticancer agent or treatment, and/or the ceramide degradation inhibitor according to claim 6, wherein the natural cannabinoid agent is a cannabigerol-type agent (CBG), and is selected from the group consisting of: cannabigerol (E) -CBG-C5; cannabigerol monomethyl ether (E)-CBGM-C₅ A; cannabinerolic acid A (Z)-CBGA-C₅; cannabigerovarine (E)-CBGV-C₃; cannabigerolic acid A (E)-CBGA-C₅; cannabigerolic acid A monomethyl ether (E)-CBGAM-C₅; cannabigerovarinic acid A (E)-CBGVA-C₃, or combinations thereof. In another preferred embodiment, the natural cannabinoid agent is a cannabichromene-type agent (BCC), and is selected from the group consisting of: (±)-cannabichromene CBC-C₅; (±)-cannabichromene acid A CBC-C₅ A; (±)-cannabichromevarin CBCV-C₃; (±)-cannabichromevarinic acid A CBCV-C₃ A, or combinations thereof.

8. The ceramide-generating anticancer agent or treatment, and/or the ceramide degradation inhibitor according to claim 6, wherein the natural cannabinoid agent is a cannabidiol-type agent (CBD), and is selected from the group consisting of: (-)-cannabidiol CBD-C₅; cannabidiol momometil ether CBDM-c₅; cannabidiol-C4 CBD-C₄; (-)-cannabidivarine CBDV-C₃; cannabidiorcol CBD-C₁; cannabidiolic acid CBDA-C₅; cannabidivarinic acid CBDVA-C₃, or combinations thereof.

9. The ceramide-generating anticancer agent or treatment, and/or the ceramide degradation inhibitor according to claim 6, wherein the natural cannabinoid agent is a cannabinodiol-type agent (CBND), and is selected from the group consisting of: cannabinodiol CBND-C₅; cannabinodivarine CBND-C₃, or combinations thereof.

10. The ceramide-generating anticancer agent or treatment, and/or the ceramide degradation inhibitor according to claim 6, wherein the natural cannabionoid agent is a tetrahydrocannabinol-type agent (THC), and is selected from the group consisting of: Δ⁹-Tetrahydrocannabinol Δ⁹-THC-C₅; Δ⁹-Tetrahydrocannabinol-C₄ Δ⁹-THC-C₄; Δ⁹-Tetrahydrocannabivarine Δ⁹-THC-C₃; Δ⁹-Tetrahydrocannabiorcol Δ⁹-THCO-C₁; Δ⁹-Tetrahydrocannabinolic acid A Δ⁹-THCA-C₅ A; Δ⁹-Tetrahydrocannabinolic acid B Δ⁹-THCA-C₅ B; Δ⁹-tetrahydrocannabinolic-C₄ A and/or B Δ⁹-THCA-C₄ and/or B; Δ⁹-tetrahydrocannabivarinic acid A Δ⁹-THCA-C₃ A; Δ⁹-tetrahydrocannabiorcolic A and/or B Δ⁹-THCOA-C₁ A and/or B; (-)-Δ⁸-trans (6a R, 10aR)-Δ⁸-tetrahydrocannabinol Δ⁸-THCA-C₅; (-)-Δ⁸-trans-(6aR,10aR)-tetrahydrocannabinolic acid Δ⁸-THCA-C₅ A; (-)-(6aS,10aR) Δ⁹-tetrahydrocannabinol (-)-cis-Δ⁹-THC-C₅, or combinations thereof.

11. The ceramide-generating anticancer agent or treatment, and/or the ceramide degradation inhibitor according to claim 6, wherein the natural cannabionoid agent is a cannabinol-type agent (CBN), and is selected from the group consisting of: cannabinol CBN-C₅; cannabinol-C₄ CBN-C₄; cannabivarin CBN-C₃; cannabinol-C₂ CBN-C₂; cannabiorcol CBN-C₁; cannabinolic acid A CBNA-C₅, or combinations thereof.

12. The ceramide-generating anticancer agent or treatment, and/or the ceramide degradation inhibitor according to claim 6, wherein the natural cannabionoid agent is a cannabitriol-type agent (CBT), and is selected from the group consisting of: (-)-(9R,10R)-trans-cannabitriol (-)-trans-CBT-C₅; (+) (9S,10S)-Cannabitriol (+)-trans-CBT-C₅; (±)-(9R,10S/9S,10R)-Cannabitriol (±)-cis-CBT-C₅; (-)-(9R, 10R)-trans-10-O-Ethylcannabitriol (-)-Trans-CBT-OEt-C₅; (±)-(9R,10R/9S,10S)-Cannabitriol-C₃ (±)-transCBT-C₃; 8,9-Dihydroxy-Δ^{6a(10a)}-tetrahydrocannabinol 8,9-Di-OH-CBT-C₅; cannabidiolic acid A cannabitriol ester CBDA-C₅ 9-OH-CBT-C₅ ester; (-)-(6aR,9S,10S,10aR)-9,10-Dihydroxy-hexahidrocannabinol, Cannabiripsol Cannabiripsol-C₅; (-)-7a,10a trihydroxy Δ⁹-tetrahydrocannabinol (-)-Cannabitetrol; 10-Oxo-Δ^{6a(10a)} tetrahydrocannabinol OTHC, or combinations thereof.

13. The ceramide-generating anticancer agent or treatment, and/or the ceramide degradation inhibitor according to claim 6, wherein the natural cannabinoid agent is a cannabinol-type agent cannabielsoin (CBE), and is selected from the group consisting of: (5AS,6S,9R,9aR)-Cannabielsoin CBE-C₅; (5AS,6S,9R,9aR)-C3-Cannabielsoin CBE -C₃; (5AS,6S,9R,9aR) Cannabielsoic acid A CBEA-C₅ A; (5AS,6S,9R,9aR)-Cannabielsoic acid CBEA-C5 B CBEA-C₅ B; Cannabiglendol-C₃ OH-iso-HHCV-C₃; Dehidrocannabifuran DCBF-C₅; Cannabifuran CBF-C₅, or combinations thereof.

14. The ceramide-generating anticancer agent or treatment, and/or the ceramide degradation inhibitor according to claim 6, wherein the natural cannabinoid agent is an isocannabinoid-type agent, and is selected from the group consisting of: (-)-Δ⁷-trans-(1R,3R,6R)-Isotetrahydrocannabinol; (±) cis-Δ⁷-1,2 (1R,3R,6S/1S,3S, 6R)-Isotetrahydrocannabivarin, (±) cis -Δ7-1,2 (1R,3R, 6S/1S,3S,6R)-Isotetrahydrocannabivarin, or combinations thereof.

15. The ceramide-generating anticancer agent or treatment, and/or the ceramide degradation inhibitor according to claim 6, wherein the natural cannabinoid agent is a cannabiciclol-type agent (CBL), and is selected from the group consisting of: (±)-(1aS,3aR,8bR,8CR) -Cannabiciclol CBL-C₅; (±)-(1aS,3aR,8bR,8CR)-Cannabiciclolic acid CBLA-C₅ A; (±)-(1aS,3aR,8bR,8CR)-Cannabiciclovarin CBLV-C₃, or combinations thereof.

16. The ceramide-generating anticancer agent or treatment, and/or the ceramide degradation inhibitor according to claim 6, wherein the natural cannabinoid agent is a cannabicitran-type agent (CBL), and more preferably is Cannabicitran CBT-C₅.

17. The ceramide-generating anticancer agent or treatment, and/or the ceramide degradation inhibitor according to claim 6, wherein the natural cannabinoid is a natural cannabichromaneme-type agent, and is selected from the group consisting of: cannabichromaneme CBCN-C₅; cannabichromaneme-C₃ CBCN-C₃; cannabicoumaronone CBCON-C₅, or combination thereof.

18. The ceramide-generating anticancer agent or treatment, and/or the ceramide degradation inhibitor according to anyone of claims 1-17, wherein the cancer or tumors of the hematopoietic and lymphoid tissues is selected from the group consisting of: acute lymphoblastic leukemia (ALL), Acute myelogenous (or myeloid) leukemia (AML), Chronic lymphocytic leukemia (CLL), Acute monocytic leukemia (AMoL), Hodgkin's lymphomas (all four subtypes), Non-Hodgkin's lymphomas (all subtypes), myelomas (multiple myeloma), or combinations thereof.

19. The ceramide-generating anticancer agent or treatment, and/or the ceramide degradation inhibitor according to anyone of claims 1-18, wherein the cancer or tumor of the hematopoietic and lymphoid tissues is the acute myelogenous (or myeloid) leukemia (AML).

20. The ceramide-generating anticancer agent or treatment, and/or the ceramide degradation inhibitor according to anyone of claims 1-18, wherein the cancer or tumor of the hematopoietic and lymphoid tissues is the acute multiple myeloma (MM).

21. A composition or a pharmaceutical form comprising a ceramide-generating anticancer agent or treatment, and/or the ceramide degradation inhibitor as described in any one of claims 1-17, for its use in the prevention, treatment, or amelioration of cancer or tumors of the hematopoietic and lymphoid tissues as described in any one of claims 1-20.

22. The composition or the pharmaceutical form according to claim 21 further comprising another active ingredient.

23. The composition or the pharmaceutical form according to any one of claims 21-22 further comprising a pharmaceutically acceptable carrier.

24. The composition or the pharmaceutical form according to any one of claims 21-22 wherein the composition is a pharmaceutical composition.

25. A method of selecting a ceramide-generating anticancer agent or treatment and/or a ceramide degradation inhibitor useful in the prevention, treatment, or amelioration of cancer or tumors of the hematopoietic and lymphoid tissues as defined in any one of claims 1-24 comprising:
a) contacting the test compound with a hematopoietic and/or lymphoid cell or cell line, and
b) detecting the expression of serine palmitoyltransferase (SPT).

26. A method of selecting a ceramide-generating anticancer agent or treatment and/or a ceramide degradation inhibitor useful in the prevention, treatment, or amelioration of cancer or tumors of the hematopoietic and lymphoid tissues as defined in any one of claims 1-24 comprising:
a) contacting the test compound with a hematopoietic and/or lymphoid cell or cell line, and
b) detecting the expression of ceramide by immunohistochemistry.
